(19) United States
(12) Patent Application Publication
(10) Pub. No.: US 2011/0033882 A1
(43) Pub. Date: Feb. 10, 2011

(54) VARIANTS OF THE BACILLUS LICHENIFORMIS ALPHA-AMYLASE

(76) Inventors: Wolfgang Aehle, Zwingenberg (DE); Neelam S. Amin, Burlingame, CA (US)

Correspondence Address: DANISCO US INC.;ATTENTION: LEGAL DEPARTMENT, 925 PAGE MILL ROAD, PALO ALTO, CA, 94304, US

(21) Appl. No.: 12/600,818

(22) PCT Filed: May 28, 2008
(86) PCT No.: PCT/US08/06768

§ 371 Date: Oct. 18, 2010

Related U.S. Application Data
(60) Provisional application No. 60/924,746, filed on May 30, 2007.

(51) Int. Cl. C12N 9/28; C12N 15/56; C12N 1/00; C12N 15/63; C12N 15/75; C12N 15/78; C12N 15/80; C12N 15/76; C12N 15/70; C12N 1/21; C12N 1/15; C11D 3/386; D06M 16/00; D06L 1/14; C12P 19/00; A23L 1/01; C12Q 1/40

(52) U.S. Cl. 435/22; 536/23.2; 435/243; 435/320.1; 435/252.3; 435/254.11; 435/252.31; 435/252.35; 435/252.33; 435/252.34; 435/202; 510/226; 510/236; 435/264; 510/320; 435/263; 8/138; 435/72; 426/61; 426/7

(57) ABSTRACT

Variants of B. licheniformis alpha-amylase advantageously exhibit improved enzymatic performance. Suitable variants include those with an altered charge distribution on the surface of the enzyme or with altered active site residues. Structural modeling can inform the choice of amino acid modifications so that modified amino acids correspond to residues found in more active alpha amylases, for example. Compositions comprising the variants are useful in methods of cleaning surfaces, laundering textiles, desizing, treating starch, e.g., liquefaction and saccharification, and hydrolyzing biofilms off various substrates.

### SEQUENCE LISTING

Also attached is a sequence listing comprising SEQ ID NOS: 1-30, which are herein incorporated by reference in their entirety.

### FIELD OF THE INVENTION

Disclosed are nucleic acids encoding polypeptides with amylase activity, wherein the polypeptide is a modified form of a Bacillus α-amylase, particularly a Bacillus licheniformis α-amylase.

### BACKGROUND

Starch consists of a mixture of amylose (15-30% w/w) and amylopectin (70-85% w/w). Amylose consists of linear chains of α-1,4-linked glucose units having a molecular weight (MW) from about 60,000 to about 800,000. Amylopectin is a branched polymer containing the same α-1,4-linked glucose units, as well as α-1,6 branch points every 24-30 glucose units; its MW may be as high as 100 million.

Sugars from starch, in the form of concentrated dextrose syrups, are currently produced by an enzyme catalyzed process involving: (1) liquefaction (or thinning) of solid starch with an α-amylase into dextrins having an average degree of polymerization of about 7-10, and (2) saccharification of the resulting liquefied starch, i.e., starch hydrolysate, with amyloglucosidase (also called glucoamylase). The resulting syrup has a high glucose content. Much of the glucose syrup that is commercially produced is subsequently enzymatically isomerized to a dextrose/fructose mixture known as isosyrup.

α-Amylases (EC 3.2.1.1) hydrolyze starch, glycogen, and related polysaccharides by cleaving internal α-1,4-glucosidic bonds at random. These enzymes have a number of important commercial applications, including starch liquefaction, textile desizing, starch modification in the paper and pulp industry, grain processing, baking and brewing. α-Amylases also can be used in automatic dishwashing detergent and laundry detergent formulations, including those containing bleach, to remove starchy stains during washing.

α-Amylases are isolated from a wide variety of bacterial, fungal, plant and animal sources. Many industrially important α-amylases are isolated from Bacillus species, e.g., Bacillus licheniformis, in part because of the high capacity of Bacillus to secrete amylases into the growth medium. While B. licheniformis α-amylase can be produced economically, the enzyme does not perform as well as other α-amylases in some applications, even though B. licheniformis α-amylase shares significant structural homology with these α-amylases. Accordingly, there is a need for B. licheniformis α-amylase variants having greater performance, especially when formulated in detergent formulations or other cleaning formulations.

### SUMMARY

Variants of B. licheniformis α-amylase are provided that have a higher performance in cleaning formulations. These B. licheniformis α-amylase variants can be used in a variety of compositions and processes which use α-amylases.

It is an object to provide an isolated nucleic acid encoding a variant of SEQ ID NO:1, wherein the variant comprises at least one amino acid substitution, insertion, or deletion compared to SEQ ID NO:1 (Van den Elzen, P., Pen, J., Hoekema, A., Sijmons, P. C., Van, Ooyen, A. J. J., Rietveld, K. and Quax, W., Transgenic plants having a modified carbohydrate content Patent: EP 0479359-A 08-APR-1992; GIST-BROCADES N.V.; MOGEN INTERNATIONAL N.V), and wherein the encoded variant exhibits α-amylase activity. The at least one amino acid substitution, insertion, or deletion may result in the encoded variant comprising an amino acid residue that corresponds to an amino acid residue of the Bacillus sp. no. 707 α-amylase set forth in SEQ ID NO:2 (Tsukamoto, A., Kimura, K., Ishii, Y., Takano, T. and Yamane, K., Nucleotide sequence of the maltohexaose-producing amylase gene from an alkalophilic Bacillus sp. #707 and structural similarity to liquefying type alpha-amylases Biochem. Biophys. Res. Commun. 151 (1), 25-31 (1988). The at least one amino acid substitution, insertion, or deletion may be made to a charged residue located on the surface of the encoded variant or to an active site amino acid residue. In one embodiment, the at least one amino acid substitution, insertion, or deletion is made to an amino acid residue other than the residue at position 1. The variant may comprises a domain A extending from residues 2-105 and residues 208-396, a domain B extending from residues 106-207, and a domain C extending from residue 397 to the C terminus. The variant may have at least one amino acid substitution, insertion, or deletion in domain A, B or C. The variant may have at least two, at least five, at least ten, 11-30, or 11-70 amino acid substitutions, insertions, or deletions. The total number of amino acid substitutions, insertions, or deletions may be 1 to 30, or 1 to 50, or 1 to 70, or any integer in between. In some embodiments, the variant has one of the amino acid sequences of SEQ ID NOS: 3-15 (SEQ ID NO. 3 is parent for variants). The variant may comprise one or more of the following amino acid substitutions, insertions, or deletions: K23N; Q26R; A33K; T49A; A52N; H68N; E82Q; K88N; H91K; R93N; D94G; D114L; T116R; D121N; A123N; D124N; R127Q; V128E; 1129V; H133Y; L134T; K136E; H140Y; H142D; S148N; Y150H; D152N; H156R; T163V; E167Q; K170R; insertion of N at position 172; Q178R; A181G; S187D; N188T; N190F; K213R; R214N; E222T; F238Y; E250S; K251A; E255N; Y262F; Q264K; H293Y; T297K; R305Q; K306N; K319H; G332E; Q333E; S334A; Q340E; T341E; substitution or deletion of residues 369-377 from TKGDSQREI to IPTHGV---, where the hyphens represent deletions; K389E; K392Q; Q393K; A398R; H400N; D416N; V419H; R437W; N444K; E447Q; H450S; E458G; E469N; or H471S.

Another object is to provide a host cell comprising the nucleic acid above. A vector comprising the nucleic acid above is also provided, as is a host cell comprising the vector. The host cell may be a microorganism, including, but not limited to, a bacterium or a fungus. The bacterial host cell may be a Gram positive bacterium selected from the group consisting of Bacillus subtilis, B. licheniformis, B. lentus, B. brevis, B. stearothermophilus, B. alkalophilus, B. amyloliquefaciens, B. coagulans, B. circulans, B. lautus, B. thuringiensis, Streptomyces lividans, or S. murinus; or a Gram negative bacterium, where the Gram negative bacterium is Escherichia coli or a Pseudomonas sp.

Another object is to provide a method of making a B. licheniformis α-amylase variant, comprising: (1) comparing the structure of a wild-type B. licheniformis α-amylase to a model α-amylase that possesses at least one preferred property relative to said wild-type B. licheniformis α-amylase; (2) identifying at least one amino acid or structural region of the wild-type B. licheniformis α-amylase that is structurally conserved with the model α-amylase; (3) constructing a variant of the wild-type B. licheniformis α-amylase, which is modified in the amino acid residue or structural region identified in step (2) above; and (4) testing the variant to determine whether the at least one preferred property is conferred upon the variant, where the variant has at least one altered property compared to the wild-type B. licheniformis α-amylase. In one embodiment, the model α-amylase is a Bacillus sp. no. 707 α-amylase.

Another object is to provide a manual or automatic dishwashing composition comprising the variant above. The manual or automatic dishwashing composition may further comprise one or more of a surfactant, detergent builder, a complexing agent, a polymer, a bleaching system, a stabilizer, a foam booster, a suds suppressor, an anti-corrosion agent, a soil-suspending agent, an anti-soil redeposition agent, a dye, a bactericide, a hydrotope, a tarnish inhibitor, and a perfume. A method of cleaning dishes comprises administering the manual or automatic dishwashing composition for a time sufficient to clean the dishes.

Another object is to provide a detergent additive comprising the variant above. A laundry detergent comprising the detergent additive further may comprise one or more of a surfactant, detergent builder, a complexing agent, a polymer, a bleaching system, a stabilizer, a foam booster, a suds suppressor, an anti-corrosion agent, a soil-suspending agent, an anti-soil redeposition agent, a dye, a bactericide, a hydrotope, an optical brightener, a fabric conditioner, and a perfume. The detergent additive may be used for laundry washing or dishwashing. The detergent additive optionally may be in the form of a non-dusting granulate, microgranulate, stabilized liquid, or protected enzyme. The detergent additive further may comprise an enzyme selected from the group consisting of: a cellulase, a protease, an aminopeptidase, an amylase, a carbohydrase, a carboxypeptidase, a catalase, a chitinase, a cutinase, a cyclodextrin glucanotransferase, a deoxyribonuclease, an esterase, an α-galactosidase, a β-galactosidase, a glucoamylase, α-glucosidase, a β-glucosidase, a haloperoxidase, an invertase, a laccase, a lipase, a mannosidase, an oxidase, a pectinolytic enzyme, a peptidoglutaminase, a peroxidase, a phytase, a polyphenoloxidase, a proteolytic enzyme, a ribonuclease, a transglutaminase, a xylanase, a pullulanase, an isoamylase, a carrageenase, or any combination thereof. The amylase may be another α-amylase, a β-amylase, an isoamylase, or a glucoamylase.

It is further an object to provide a detergent composition comprising the detergent additive above or the variant above. The detergent composition further may comprise an enzyme from the group consisting of: a cellulase, a protease, an aminopeptidase, an amylase, a carbohydrase, a carboxypeptidase, a catalase, a chitinase, a cutinase, a cyclodextrin glucanotransferase, a deoxyribonuclease, an esterase, an α-galactosidase, a β-galactosidase, a glucoamylase, an α-glucosidase, a β-glucosidase, a haloperoxidase, an invertase, a laccase, a lipase, a mannosidase, an oxidase, a pectinolytic enzyme, a peptidoglutaminase, a peroxidase, a phytase, a polyphenoloxidase, a proteolytic enzyme, a ribonuclease, a transglutaminase, a xylanase, a pullulanase, an isoamylase, a carrageenase, or any combination thereof.

It is yet another object to provide a textile desizing composition comprising the variant above in an aqueous solution, and optionally with another enzyme. A method of desizing a textile comprises administering the desizing composition for a time sufficient to desize said textile.

Another object is to provide a starch processing composition comprises the variant above in an aqueous solution. The starch processing composition further may comprise a glucoamylase, an isoamylase, a pullulanase, phytase or a combination thereof. A method of processing a starch comprises administering the composition for a time sufficient to process the starch.

Another object is to provide a biofilm hydrolyzing composition comprising the variant above in a solution or gel, and optionally with a cellulase, a hemicellulase, a xylanase, a lipase, a protease, a pectinase, an antimicrobial agent, or any combination thereof. A method of hydrolyzing a biofilm comprises administering the composition for a period sufficient to process the biofilm.

Another object is to provide a composition for saccharifying starch comprising the variant in a solution. A method of saccharifying starch comprises administering the composition for a period sufficient to saccharify the starch.

Another object is to provide a composition for liquefying starch comprising the variant above in a solution. A method of liquefying a starch comprises administering the composition for a period sufficient to liquefy the starch.

Another object is to provide a baking composition comprising the variant above in a solution or in a gel. A method of baking comprises administering the baking composition to a substance to be baked.

### BRIEF DESCRIPTION OF THE DRAWINGS

The accompanying drawings are incorporated in and constitute a part of this specification, illustrate embodiments. In the drawings:

FIG. 1 depicts a 3D structural alignment between a B. licheniformis α-amylase used in PURASTAR® OxAm (Danisco US Inc., Genencor Division; previously known as Genencor International, Inc.) and a B. subtilus sp. no. 707 α-amylase (Swissprot Accession No. P19571). The structural alignment includes a substrate analogue Acarbose (an inhibitor) shown bound to the active site. The A-domain of the α-amylases is located in the middle of the aligned structures to the right of the substrate, the B-domain is on the left hand side to the left of the substrate, and the C-domain occupies the right side of the picture.

FIG. 2 depicts a sequence alignment between the wild-type B. licheniformis α-amylase (Accession No. CAA01355; top line) and Bacillus sp. no. 707 α-amylase (Swissprot Accession No. P19571; bottom line). Identical residues are marked underneath with an asterix symbol.

FIG. 3 depicts the number, general type and/or domain location of the amino acid substitutions, insertions or deletions for twelve B. licheniformis α-amylase variants.

FIG. 4 depicts a diagram of plasmid pHPLT-OxAm used for the expression of OxAm parent and variants.

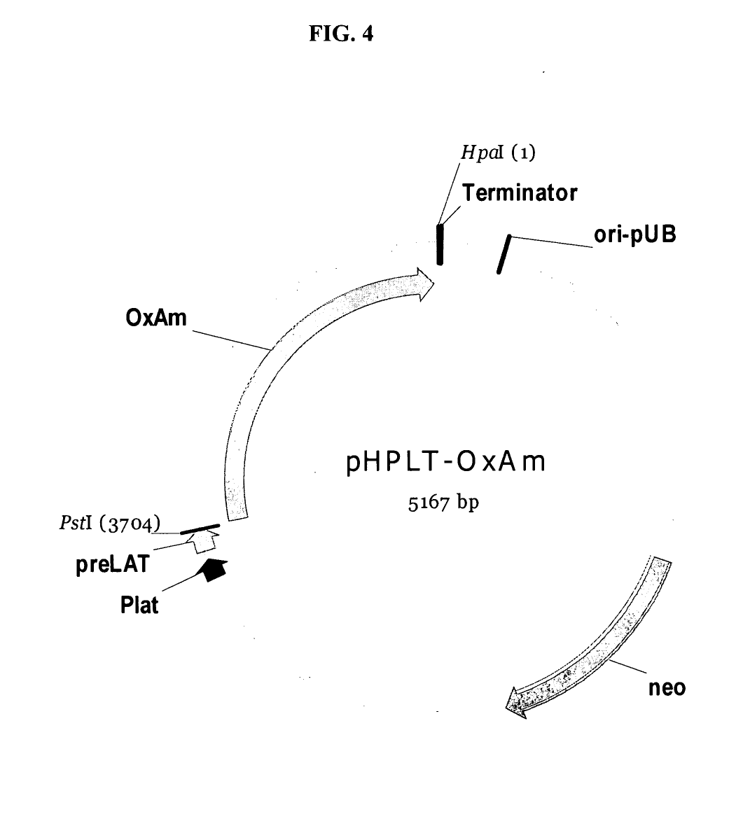

FIG. 5 depicts the cleaning activity of OxAm and OxAm variants (V2, V3, and V5) grown in either cultivation media M1 or M2.

FIG. 6 depicts the cleaning activity of OxAm and OxAm variants (V1, V2, V3, V5, V6, and V9) grown in cultivation medium M1.

### DETAILED DESCRIPTION

Variants of B. licheniformis α-amylase are provided with greater performance in cleaning formulations, such as automatic dishwashing detergent and laundry detergent formulations, including those containing bleach. In particular, the present variants have a higher specific activity than wild-type B. licheniformis α-amylase. The following provides details on how this can be done, as well as compositions and uses for the α-amylase variants produced thereby.

### 1. DEFINITIONS & ABBREVIATIONS

In accordance with this detailed description, the following abbreviations and definitions apply. It should be noted that as used herein, the singular forms “a,” “an,” and “the” include plural referents unless the context clearly dictates otherwise. Thus, for example, reference to “an enzyme” includes a plurality of such enzymes and reference to “the formulation” includes reference to one or more formulations and equivalents thereof known to those skilled in the art, and so forth.

Unless defined otherwise, all technical and scientific terms used herein have the same meaning as commonly understood by one of ordinary skill in the art. The following terms are provided below.

### 1.1. Definitions

“Amylase” means an enzyme that is, among other things, capable of catalyzing the degradation of starch. “Amylase” includes any amylase, such as glucoamylases, α-amylase, β-amylases, and wild-type α-amylases of Bacillus sp., such as B. licheniformis and B. subtilis. Amylases are hydrolases that cleave the α-D-(1→4) O-glycosidic linkages in starch. Generally, α-amylases (EC 3.2.1.1; α-D-(1→4)-glucan glucanohydrolase) are defined as endo-acting enzymes cleaving α-D-(1→4) O-glycosidic linkages within the starch molecule in a random fashion. In contrast, the exo-acting amylolytic enzymes, such as β-amylases (EC 3.2.1.2; α-D-(1→4)-glucan maltohydrolase) and some product-specific amylases like maltogenic α-amylase (EC 3.2.1.133) cleave the starch molecule from the non-reducing end of the substrate. β-Amylases, α-glucosidases (EC 3.2.1.20; α-D-glucoside glucohydrolase), glucoamylase (EC 3.2.1.3; α-D-(1→4)-glucan glucohydrolase), and product-specific amylases can produce malto-oligosaccharides of a specific length from starch.

“α-Amylase variant,” “α-amylase variant polypeptide,” and “variant enzyme” mean an α-amylase protein that has an amino acid sequence that has been modified from the amino acid sequence of a wild-type α-amylase. As used herein, “parent enzymes,” “parent sequence,” “parent polypeptide,” “wild-type α-amylase protein,” and “parent polypeptides” mean enzymes and polypeptides from which the α-amylase variant polypeptides are based. A wild-type α-amylase occurs naturally. For the purpose of this disclosure, the B. licheniformis α-amylase used in PURASTAR® OxAm is considered a “wild-type” α-amylase. That is, a “B. licheniformis α-amylase variant” specifically excludes the B. licheniformis α-amylase used in PURASTAR® OxAm. “PURASTAR® OxAm,” “PURASTAR®” and “OxAm” are used interchangeably herein. “α-Amylase variant” also specifically excludes α-amylases that differ from a wild-type α-amylase only in the amino acid residues of the signal sequence or the first residue of the mature protein. That is, for the purpose of this disclosure, the sequence of the mature α-amylase variant differs from the sequence of a mature wild-type α-amylase at a position other than the first residue.

“Variants” refer to both polypeptides and nucleic acids. The term “variant” may be used interchangeably with the term “mutant.” Variants include insertions, substitutions, transversions, truncations, and/or inversions at one or more locations in the amino acid or nucleotide sequence, respectively. Variant nucleic acids can include sequences that are complementary to sequences that are capable of hybridizing to the nucleotide sequences presented herein. For example, a variant sequence is complementary to sequences capable of hybridizing under stringent conditions (e.g., 50° C. and 0.2×SSC {1×SSC=0.15 M NaCl, 0.015 M sodium citrate, pH 7.0}) to the nucleotide sequences presented herein. More particularly, the term variant encompasses sequences that are complementary to sequences that are capable of hybridizing under highly stringent conditions (e.g., 65° C. and 0.1×SSC) to the nucleotide sequences presented herein.

“Isolated” means that the sequence is at least substantially free from at least one other component that the sequence is naturally associated and found in nature.

“Purified” means that the material is in a relatively pure state, e.g., at least about 90% pure, at least about 95% pure, or at least about 98% pure.

“Thermostable” means the enzyme is more thermostable than a reference enzyme. In the present application, an α-amylase variant is more thermostable than a wild-type B. licheniformis α-amylase if the variant has a relatively higher enzymatic activity after a specific interval of time under the same experimental conditions, e.g., the same temperature, substrate concentration, etc. Alternatively, a more thermostable enzyme has a higher heat capacity determined by differential scanning calorimetry, compared to a reference enzyme.

“pH range” means the pH values over which an enzyme exhibits activity.

As used herein, “pH stable” means the enzyme is more stable than a reference enzyme at a particular pH. In the present application, an α-amylase variant is more pH stable than a wild-type B. licheniformis α-amylase if the variant has a relatively higher activity after a specific interval of time under the same experimental conditions, e.g., the same pH, etc.

As used herein, “food” includes both prepared food, as well as an ingredient for a food, such as flour.

As used herein, “food ingredient” includes a formulation that is or can be added to a functional food or foodstuff and includes formulations used at low levels in a wide variety of products that require, for example, acidifying or emulsifying. The food ingredient may be in the form of a solution or as a solid, depending on the use and/or the mode of application and/or the mode of administration.

As used herein, “functional food” means food capable of providing not only a nutritional effect and/or a taste satisfaction, but also any further beneficial effect to the consumer.

As used herein, “amino acid sequence” is synonymous with the term “polypeptide” and/or the term “protein.” In some instances, the term “amino acid sequence” is synonymous with the term “peptide”; in some instances, the term “amino acid sequence” is synonymous with the term “enzyme.”

As used herein, “nucleotide sequence” or “nucleic acid sequence” refers to an oligonucleotide sequence or polynucleotide sequence and variants, homologues, fragments and derivatives thereof. The nucleotide sequence may be of genomic, synthetic or recombinant origin and may be double-stranded or single-stranded, whether representing the sense or anti-sense strand. As used herein, the term “nucleotide sequence” includes genomic DNA, cDNA, synthetic DNA, and RNA.

“Homologue” means an entity having a certain degree of identity or “homology” with the subject amino acid sequences and the subject nucleotide sequences. A “homologous sequence” includes an amino acid sequence at least 75%, 80%, 85% or 90% identical, particularly at least 95%, 96%, 97%, 98% or 99% identical to the subject sequence. Typically, homologues will comprise the same active site residues as the subject amino acid sequence.

As used herein, “hybridization” includes the process by which a strand of nucleic acid joins with a complementary strand through base pairing, as well as the process of amplification as carried out in polymerase chain reaction (PCR) technologies. The α-amylase variant nucleic acid may exist as single- or double-stranded DNA or RNA, an RNA/DNA heteroduplex or an RNA/DNA copolymer. As used herein, “copolymer” refers to a single nucleic acid strand that comprises both ribonucleotides and deoxyribonucleotides. The α-amylase variant nucleic acid may be codon-optimized to further increase expression.

As used herein, a “synthetic” compound is produced by in vitro chemical or enzymatic synthesis. It includes, but is not limited to, α-amylase variant nucleic acids made with optimal codon usage for host organisms, such as the methylotrophic yeasts Pichia, Hansenula, Streptomyces, and Trichoderma reesei, or other expression hosts of choice.

As used herein, “transformed cell” includes cells that have been transformed by use of recombinant DNA techniques. Transformation typically occurs by insertion of one or more nucleotide sequences into a cell. The inserted nucleotide sequence may be a heterologous nucleotide sequence, i.e., is a sequence that is not natural to the cell that is to be transformed, such as a fusion protein.

As used herein, “operably linked” means that the described components are in a relationship permitting them to function in their intended manner. For example, a regulatory sequence operably linked to a coding sequence is ligated in such a way that expression of the coding sequence is achieved under condition compatible with the control sequences.

As used herein, “biologically active” refers to a sequence having a similar structural, regulatory or biochemical function as the naturally occurring sequence, although not necessarily to the same degree.

### 1.2. Abbreviations

The following abbreviations apply unless indicated otherwise:
AE alcohol ethoxylate
AEO alcohol ethoxylate
AEOS alcohol ethoxysulfate
AES alcohol ethoxysulfate
AFAU acid fungal α-amylase units
AGU glucoamylase activity unit
AOS α-olefinsulfonate
AS alcohol sulfate
BAA bacterial α-amylase
cDNA complementary DNA
CMC carboxymethylcellulose
DE Dextrose Equivalent
DNA deoxyribonucleic acid
DP3 degree of polymerization with three subunits
DPn degree of polymerization with n subunits
DS dry solid
DTMPA diethyltriaminepentaacetic acid
EC enzyme commission for enzyme classification
EDTA ethylenediaminetetraacetic acid
EDTMPA ethylenediaminetetramethylene phosphonic acid
EO ethylene oxide
F&HC fabric and household care
HFCS high fructose corn syrup
HFSS high fructose starch based syrup
IPTG isopropyl β-D-thiogalactoside
LAS linear alkylbenezenesulfonate
LU Lipase Units
MW molecular weight
nm nanometer
NOBS nonanoyloxybenzenesulfonate
NTA nitrilotriacetic acid
PCR polymerase chain reaction
PEG polyethyleneglycol
pI isoelectric point
ppm parts per million
PVA poly(vinyl alcohol)
PVP poly(vinylpyrrolidone)
RAU Reference Amylase Units
RMS root mean square
RNA ribonucleic acid
SAS secondary alkane sulfonates
1×SSC 0.15 M NaCl, 0.015 M sodium citrate, pH 7.0
SSF simultaneous saccharification and fermentation
TAED tetraacetylethylenediamine
TNBS trinitrobenzenesulfonic acid
w/v weight/volume
w/w weight/weight
wt wild-type
μL microliter

### 2. α-AMYLASE VARIANTS

The α-amylase variants herein are created from wild-type B. licheniformis α-amylase. The present variants may have enhanced specific activity, pH profile, thermostability, temperature range profile, calcium ion requirements, or other enhanced characteristics. Variants generally contain one or more modifications of the amino acid sequence of a wild-type B. licheniformis α-amylase. A wild-type B. licheniformis α-amylase may be isolated from any naturally occurring strain of B. licheniformis.

For the purpose of this disclosure, an amino acid substitution may be designated M15T, for instance. “M15T” means that a methionine (M) residue at position 15 is replaced with a threonine (T) residue, where the amino acids are designated by single letter abbreviations commonly known in the art.

Protein engineering of a wild-type B. licheniformis α-amylase generates variant α-amylases that can have improved properties. In one aspect, one or more amino acid residues of the variant enzyme are modified randomly, and the effect of the modifications is determined by subsequent analysis of the performance characteristics of the variant, following host cell expression of the variant. In another aspect, modifications to the amino acid sequence of the variant are made systematically, using a “model” α-amylase having a structure very similar to the wild-type B. licheniformis α-amylase as a guide, so that the effect of the modifications can be predicted. In one embodiment, the model α-amylase has one or more characteristics that are improved or preferred with respect to the wild-type B. licheniformis α-amylase. For example, the model α-amylase may have a higher specific activity, pH dependence, stability, half-life, or calcium binding constant, or it may have a particularly useful substrate specificity, etc.

If a model α-amylase is used to guide the design of amino acid changes of the variant α-amylase, it is not necessary to know precisely which residues of the model α-amylase contribute to the performance of the enzyme. Instead, one or more amino acids, even an entire set of amino acids, are modified in the variant α-amylase to the corresponding amino acid(s) of the model α-amylase. A “corresponding” amino acid in this case is not determined by a conventional alignment of the primary amino acid sequence, but by a 3D structural alignment of the polypeptide backbone of the two enzymes. Amino acids to be modified in the variant thus can be chosen as charged residues on the enzyme surface, active site residues, or residues that contribute to particular secondary structural elements unique to the model enzyme, for example. The residues to be modified also can be selected on the basis that the modification would not disrupt conserved 3D structures between the two enzymes, particularly conserved secondary structural elements, e.g., α-helices, β-sheets, turns.

For example, it is known that changing the distribution of charged amino acids on the surface of an enzyme generally can alter its enzymatic properties. See, e.g., Russell et al., “Rational modification of enzyme catalysis by engineering surface charge,” Nature 328: 496-500 (1987). One or more residues on the surface of the B. licheniformis α-amylase likewise can be modified to alter the enzymatic properties of the variant α-amylase, where the choice of modifications can be guided by the distribution of surface charges on the model α-amylase. For this purpose, a “surface charge” is contributed by a charged side chain of an amino acid that is at least partially exposed to solvent.

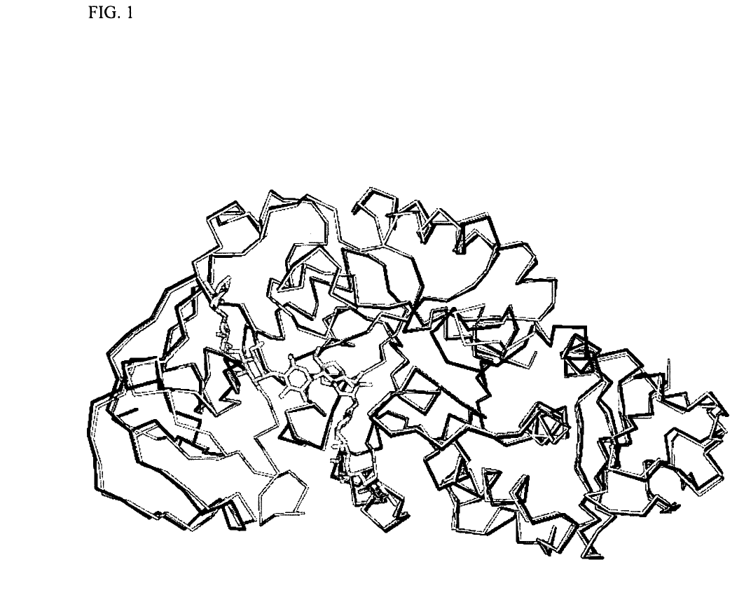

FIG. 1 shows a 3D structural alignment of B. licheniformis α-amylase (see RCSB Protein Data Bank, Accession No. PDB ID No. 1BLI; see also GenBank Accession No. CAA01355) with a representative model α-amylase, B. subtilus sp. no. 707 α-amylase. See Berman et al., “The Protein Data Bank,” Nucl. Acids Res. 28: 235-242 (2000). For the analysis, three amino acids were changed from the sequence used to build the 3D structure shown in Accession No. PDB ID No. 1BLI, namely M15T, W138Y, and M197T, so that the structure would be identical to the B. licheniformis α-amylase used in PURASTAR® OxAm. The 3D structure of B. subtilus sp. no. 707 α-amylase was accessed from the RCSB Protein Data Bank as Accession NO. PDB ID No. 1WPC (see also Swissprot Accession No. P19571).

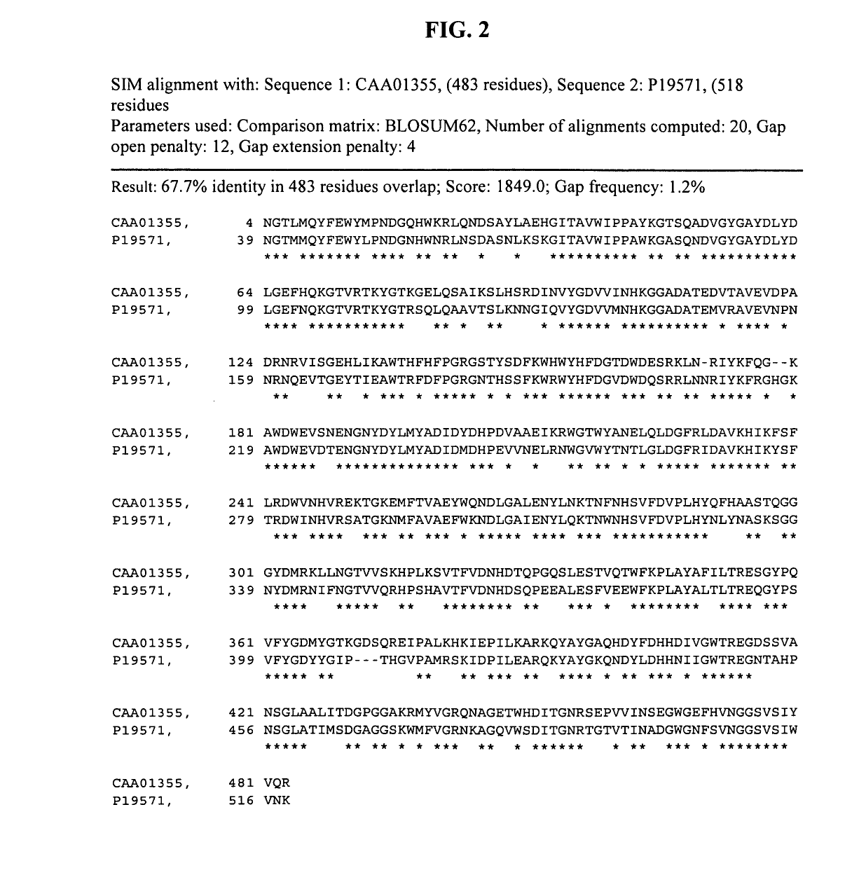

Alignment algorithms, such as BRAGI (Gesellschaft für Biotechnologische Forschung mbH) or PyMOL (DeLano Scientific LLC), can be used to obtain a best fit between the 3D structures of the two enzymes. These programs iteratively align the backbone atoms of the two molecules to minimize the root mean square (RMS) deviation of the spatial distances of the atomic positions. A representative output from a protein sequence alignment is shown in FIG. 2, which shows a B. licheniformis α-amylase sequence (GenBank Accession No. CAA01355) on the bottom row and a B. subtilus sp. no. 707 α-amylase (Swissprot Accession No. P19571) on the top row. The asterisks in the line between the two sequences mark residues that contain backbone atoms that adopt substantially the same 3D-structure. The two enzymes show strong structural conservation in secondary structures of the enzymes, such as α-helices, β-sheets, turns, etc. Over the entire molecules, the RMS of the deviation between the enzymes is 0.55 Å, which is less than half of the margin of error in determining the crystal structure of the two enzymes. As seen in FIG. 2, 93% of the residues adopt the same 3D structure between the two enzymes, which exceeds the percentage identity in primary sequence between the two enzymes (identical residues are highlighted). For the purpose of this disclosure, residues that adopt the same 3D structure are “structurally conserved,” and “structurally conserved” residues are “corresponding” residues in the two structures.

A residue of the variant α-amylase can be classified as belonging to one of three structural domains, herein called domains A, B and C. For the purpose of this disclosure, domain A extends from residues 2-105 and from residues 208-396; domain B extends from residues 106-207; and domain C extends from residue 397 to the C terminus of the protein. An amino acid also can be classified as an active site residue. Active site residues are located at least at positions 49, 52, 163, 167, 170, 172, 187, 188, 190, 238, 262, 264, 293, 297, and 332-334. Residue “positions” are numbered as depicted in the B. licheniformis α-amylase sequence in FIG. 2.

In the variant α-amylase, one or more amino acid can be modified to the corresponding amino acid in the model α-amylase. The modifications may be clustered by domain, and/or they may be clustered by amino acids that are charged and present on the surface of the enzyme. Alternatively or in addition, modifications may be made to one or more active site residues. In this manner, it is possible to make multiple amino acid modifications, where the modifications have a predictable effect on the performance characteristics of the variant α-amylase. For example, the variant may have every surface charged residue in one or more domain changed to the corresponding residue of the model α-amylase. In another embodiment, the variant may have residues inserted or deleted, e.g., a loop may be inserted or deleted, such that the polypeptide backbone of the variant more closely resembles the structure of the model α-amylase. Accordingly, the variant may comprise 1, 2, 3, 4, 5, 10, 15, 20, 30, 40, 50, 60 or 70 amino acid substitutions, deletions or insertions, or any integer value in between, provided the variant retains α-amylase activity. The surface charge of the variant also may be altered by any number. For example, the number of positively charged amino acid residues on the enzyme surface may be reduced by 1, 2, 3, 4, 5, 6, 7 or 8. Such amino acid substitutions are expected to change the isoelectric point (pI) of the variant, among other things. Other characteristics of the variant may differ from the wild-type enzyme, as described below.

Accordingly, a method for making a B. licheniformis α-amylase variant is provided, where the variant has at least one altered property compared to the wild-type B. licheniformis α-amylase. The method comprises: (1) comparing the structure of the wild-type α-amylase to a model α-amylase that possesses at least one preferred property relative to the wild-type α-amylase; (2) identifying at least one amino acid or structural region of the wild-type B. licheniformis α-amylase that is structurally conserved with the model α-amylase; (3) constructing a variant of the B. licheniformis α-amylase, which is modified in the amino acid residue or structural part identified in step (2) above; and (4) testing the resulting variant α-amylase to determine whether the at least one preferred property is conferred upon the variant α-amylase.

The structure identified in step (2) of the method above may be composed of one amino acid residue; however, the structure also may comprise more than one amino acid residue, located in one or more the A, B, or C domains and/or the active site of the enzyme. The more than one amino acids may be contiguous, as where a loop of several amino acids is either added or deleted from the variant α-amylase, for example. The modification of an amino acid residue or structural region is typically accomplished by suitable modifications of a DNA sequence encoding the parent enzyme in question. The modification may be substitution, deletion or insertion of an amino acid residue or a structural part.

In one embodiment, the amino acid substitution, deletion or insertion may be one or more of the following, or any combination thereof: K23N; Q26R; A33K; T49A; A52N; H68N; E82Q; K88N; H91K; R93N; D94G; D114L; T116R; D121N; A123N; D124N; R127Q; V128E; 1129V; H133Y; L134T; K136E; H140Y; H142D; S148N; Y150H; D152N; H156R; T163V; E167Q; K170R; an insertion of N at position 172 (i.e., 172+N); Q178R; A181G; S187D; N188T; N190F; K213R; R214N; E222T; F238Y; E250S; K251A; E255N; Y262F; Q264K; H293Y; T297K; R305Q; K306N; K319H; G332E; Q333E; S334A; Q340E; T341E; a substitution or deletion of residues at positions 369-377 from TKGDSQREI to IPTHGV---, where the hyphens represent deletions; K389E; K392Q; Q393K; A398R; H400N; D416N; V419H; R437W; N444K; E447Q; H450S; E458G; E469N; or H471S. “K23N,” for example, means that the lysine (K) residue at position 23 of the B. licheniformis α-amylase having the sequence shown in the bottom row of FIG. 2 is substituted with an asparagine (N) residue in the variant.

The α-amylase variant can also be a fusion protein, or “hybrid” or “chimeric protein,” comprising a polypeptide sequence not endogenous to B. licheniformis. In one embodiment, the polypeptide sequence facilitates purification of the expressed protein. In another embodiment, the heterologous sequence is an α-amylase polypeptide derived from a different genus or species than B. licheniformis. For example, the α-amylase variant can comprise a variant of a B. licheniformis α-amylase linked to the signal peptide of another Bacillus α-amylase, such as, but not limited to, B. stearothermophilus.

2.1. α-Amylase Variant Characterization

Enzyme variants can be characterized by nucleic acid and polypeptide sequences, by their 3D structures as described above, and/or by their specific activity. Additional features of the α-amylase variant include stability, calcium ion (Ca²⁺) dependence, pH range, oxidation stability, and thermostability. In one aspect, the α-amylase variants in cleaning formulations have higher specific activities, which can be assessed using standard assays known to the artisan skilled in this field. In another aspect, variants demonstrate other improved performance characteristics, such as improved stability at high temperatures (i.e., 70-120° C.), and/or pH extremes (i.e., pH 4.0 to 6.0 or pH 8.0 to 11.0), and/or calcium concentrations below 60 ppm.

Altered Ca²⁺ stability means the stability of the enzyme under Ca²⁺ depletion has been altered, i.e., increased or decreased. Mutations of importance include those that alter Ca²⁺ stability, in particular improved Ca²⁺ stability at high pH, i.e., pH 8.0 to 10.5.

In a further aspect, important mutations exhibit altered specific activity, especially at temperatures from 10-60° C., particularly 20-50° C., and more particularly 30-40° C., for use in cleaning compositions. For baking products, important mutations may exhibit altered specific activity at higher temperature ranges.

α-Amylase variants also may have altered oxidation stability, in particular higher oxidation stability, in comparison to the parent α-amylase. For example, increased oxidation stability is advantageous in detergent compositions, and decreased oxidation stability may be advantageous in composition for starch liquefaction.

The variant α-amylase may be more thermostable than the wild-type α-amylase. Such α-amylase variants are advantageous for use in baking or other processes that require elevated temperatures. For example, a thermostable α-amylase variant can degrade starch at temperatures of about 55° C. to about 80° C. or more. A thermostable α-amylase variant may retain its activity after exposure to temperatures of up to about 95° C.

The α-amylase variant polypeptides described herein can also have mutations that extend half-life relative to the parent enzyme by 10%, 20%, 30%, 40%, 50%, 60%, 70%, 80%, 90%, 100%, 200% or more, particularly at elevated temperatures of about 55° C. to about 95° C. or more, particularly at about 80° C. In one embodiment, the α-amylase variant can be heated for about 1-10 minutes at 80° C. or higher.

The α-amylase variants may have exo-specificity, measured by exo-specificity indices described herein, for example. α-Amylase variants include those having higher or increased exo-specificity compared to the parent enzymes or polypeptides from which they were derived, optionally when measured under identical conditions. Thus, for example, the α-amylase variant polypeptides may have an exo-specificity index 10%, 20%, 30%, 40%, 50%, 60%, 70%, 80%, 90%, 100%, 150%, 200%, 500%, 1000%, 5000%, 10,000% or higher compared to their parent polypeptides.

In one aspect, the α-amylase variant polypeptide encoded by the nucleic acid has the same pH stability as the parental sequence. In another aspect, the variant comprises a mutation that confers a greater pH stability range or shifts the pH range to a desired area for the end commercial purpose of the enzyme. For example, in one embodiment, the variant can degrade starch at about pH 5.0 to about pH 10.5. The α-amylase variant polypeptide may have a longer half-life or higher activity (depending on the assay) compared to the parent polypeptide under identical conditions, or the α-amylase variant may have the same activity as the parent polypeptide. The α-amylase variant polypeptide also may have about 10%, 20%, 30%, 40%, 50%, 60%, 70%, 80%, 90%, 100%, 200% or longer half-life compared to their parent polypeptide under identical pH conditions. Alternatively, or in addition, the enzyme variant may have higher specific activity compared to the parent polypeptide under identical pH conditions.

In another aspect, a nucleic acid complementary to a nucleic acid encoding any of the α-amylase variants set forth herein is provided. Additionally, a nucleic acid capable of hybridizing to the complement is provided. In another embodiment, the sequence for use in the methods and compositions described here is a synthetic sequence. It includes, but is not limited to, sequences made with optimal codon usage for expression in host organisms, such as the methylotrophic yeasts Pichia and Hansenula.

### 3. PRODUCTION of α-AMYLASE VARIANTS

A DNA sequence encoding the enzyme variant produced by methods described herein, or by any alternative methods known in the art, can be expressed, in enzyme form, using an expression vector which typically includes control sequences encoding a suitable promoter, operator, ribosome binding site, translation initiation signal, and, optionally, a repressor gene or various activator genes.

### 3.1. Vectors

The recombinant expression vector carrying the DNA sequence encoding an α-amylase variant may be any vector that may conveniently be subjected to recombinant DNA procedures, and the choice of vector will often depend on the host cell into which it is to be introduced. Thus, the vector may be an autonomously replicating vector, i.e., a vector that exists as an extrachromosomal entity, the replication of which is independent of chromosomal replication, e.g., a plasmid, a bacteriophage or an extrachromosomal element, mini-chromosome or an artificial chromosome. Alternatively, the vector may be one which, when introduced into a host cell, is integrated into the host cell genome and replicated together with the chromosome(s) into which it has been integrated. The integrated gene may also be amplified to create multiple copies of the gene in the chromosome by use of an amplifiable construct driven by antibiotic selection or other selective pressure, such as an essential regulatory gene or by complementation of an essential metabolic pathway gene.

An expression vector typically includes the components of a cloning vector, e.g., an element that permits autonomous replication of the vector in the selected host organism and one or more phenotypically detectable markers for selection purposes. The expression vector normally comprises control nucleotide sequences encoding a promoter, operator, ribosome binding site, translation initiation signal and optionally, a repressor gene or one or more activator genes. In one aspect, all the signal sequences used target the material to the cell culture media for easier enzyme collection and optionally purification. The procedures used to ligate the DNA construct encoding an α-amylase variant, the promoter, terminator and other elements, respectively, and to insert them into suitable vectors containing the information necessary for replication, are well known to persons skilled in the art (see e.g., Sambrook et al., MOLECULAR CLONING: A LABORATORY MANUAL, 2^{nd} ed., Cold Spring Harbor, 1989 and 3^{rd} ed., 2001).

In the vector, the DNA sequence should be operably connected to a suitable promoter sequence. The promoter may be any DNA sequence that shows transcriptional activity in the host cell of choice and may be derived from genes encoding proteins either homologous or heterologous to the host cell. Examples of suitable promoters for directing the transcription of the DNA sequence encoding an α-amylase variant, especially in a bacterial host, are the promoter of the lac operon of E. coli, the Streptomyces coelicolor agarase gene dagA or celA promoters, the promoters of the Bacillus licheniformis α-amylase gene (amyL), the promoters of the Bacillus stearothermophilus maltogenic amylase gene (amyM), the promoters of the Bacillus amyloliquefaciens α-amylase (amyQ), the promoters of the Bacillus subtilis xylA and xylB genes, etc. For transcription in a fungal host, examples of useful promoters are those derived from the gene encoding Aspergillus oryzae TAKA amylase, Rhizomucor miehei aspartic proteinase, Aspergillus niger neutral α-amylase, A. niger acid stable α-amylase, A. niger glucoamylase, Rhizomucor miehei lipase, A. oryzae alkaline protease, A. oryzae triose phosphate isomerase, or A. nidulans acetamidase. When the gene encoding the α-amylase variant polypeptide is expressed in a bacterial species such as E. coli, a suitable promoter can be selected, for example, from a bacteriophage promoter including a T7 promoter and a phage lambda promoter. Examples of suitable promoters for the expression in a yeast species include but are not limited to the Gal 1 and Gal 10 promoters of Saccharomyces cerevisiae and the Pichia pastoris AOX1 or AOX2 promoters. For expression in Trichoderma reesei, the CBHII promoter also may be used.

The expression vector may also comprise a suitable transcription terminator and, in eukaryotes, polyadenylation sequences operably connected to the DNA sequence encoding the α-amylase variant. Termination and polyadenylation sequences may suitably be derived from the same sources as the promoter. The vector may further comprise a DNA sequence enabling the vector to replicate in the host cell in question. Examples of such sequences are the origins of replication of plasmids pUC19, pACYC177, pUB110, pE194, pAMB1, pICatH, and pIJ702.

The vector may also comprise a selectable marker, e.g., a gene the product of which complements a defect in the host cell, such as the dal genes from B. subtilis or B. licheniformis, or a gene which confers antibiotic resistance, e.g., ampicillin, kanamycin, chloramphenicol or tetracyclin resistance. Furthermore, the vector may comprise Aspergillus selection markers such as amdS, argB, niaD and xxsC, a marker giving rise to hygromycin resistance, or the selection may be accomplished by co-transformation as known in the art. See, e.g., WO 91/17243.

### 3.2. Variant Expression and Host Organisms

While intracellular expression or solid state fermentation may be advantageous in some respects, e.g., when using certain bacteria or fungi as host cells, it is generally advantageous if the expression of the variant is extracellular and into the culture medium. In general, the Bacillus α-amylases mentioned herein comprise a signal sequence that permits secretion of the expressed protease into the culture medium. If desirable, this signal sequence may be replaced by a different signal sequence, which is conveniently accomplished by substitution of the DNA sequences encoding the respective signal sequence. The signal sequences are typically characterized as having three domains, an N-terminal domain, a H-domain, and a C-terminal domain and range from 18 to 35 residues in length.

The mature protein can be in the form initially of a fusion protein to a pre-protein derived from another Bacillus sp. or from the same species as the parental sequence. To secrete proteins in B. licheniformis, the signal peptide of B. licheniformis α-amylase is frequently used; however, signal proteins from other Bacillus α-amylases can also be substituted.

An isolated cell, either comprising a DNA construct or an expression vector, is advantageously used as a host cell in the recombinant production of an α-amylase variant. The cell may be transformed with the DNA construct encoding the variant, conveniently by integrating the DNA construct (in one or more copies) in the host chromosome. This integration is generally considered to be an advantage as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector as described above in connection with the different types of host cells.

Examples of suitable bacterial host organisms are Gram positive bacterial species such as Bacillaceae, including B. subtilis, B. licheniformis, B. lentus, B. brevis, B. stearothermophilus, B. alkalophilus, B. amyloliquefaciens, B. coagulans, B. lautus, B. megaterium, and B. thuringiensis; Streptomyces sp., such as S. murinus; lactic acid bacterial species including Lactococcus sp., such as L. lactis; Lactobacillus sp. including L. reuteri; Leuconostoc sp.; Pediococcus sp.; and Streptococcus sp. Alternatively, strains of a Gram negative bacterial species belonging to Enterobacteriaceae, including E. coli, or to Pseudomonadaceae can be selected as the host organism.

A suitable yeast host organism can be selected from biotechnologically relevant yeasts species, such as, but not limited to, Pichia sp., Hansenula sp., Kluyveromyces sp., Yarrowinia sp., Saccharomyces sp., including S. cerevisiae, or a species belonging to Schizosaccharomyces, such as S. pombe. A strain of the methylotrophic yeast species Pichia pastoris can be used as the host organism. Alternatively, the host organism can be a Hansenula species. Suitable host organisms among filamentous fungi include species of Aspergillus, e.g., A. niger, A. oryzae, A. tubigensis, A. awamori, or A. nidulans. Alternatively, a strain of Fusarium sp., e.g., Fusarium oxysporum or Rhizomucor sp., such as R. miehei, can be used as the host organism. Other suitable yeasts include Thermomyces sp. and Mucor sp. Fungal cells may be transformed by a process involving protoplast formation and transformation of the protoplasts followed by regeneration of the cell wall in a manner known in the art. A suitable procedure for transforming Aspergillus host cells, for example, is described in EP 238023.

In a yet further aspect, a method of producing an α-amylase variant is provided, which method comprises cultivating a host cell as described above under conditions conducive to the production of the variant and recovering the variant from the cells and/or culture medium. The medium used to cultivate the cells may be any conventional medium suitable for growing the host cell in question and obtaining expression of the α-amylase variant. Suitable media and media components are available from commercial suppliers or may be prepared according to published recipes, e.g., as described in catalogues of the American Type Culture Collection (ATCC). Exemplary culture media include but are not limited to those for fed-batch fermentations performed in a three thousand liter (3,000 L) stirred tank fermentor, which was used in the examples provided infra. The media used would be that most suitable for the host cell being used, for example the media discussed below for culturing Bacillus licheniformis. The growth medium in that case can consist of corn steep solids and soy flour as sources of organic compounds, along with inorganic salts as a source of sodium, potassium, phosphate, magnesium and sulfate, as well as trace elements. Typically, a carbohydrate source such as glucose is also part of the initial medium. Once the culture has established itself and begins growing, the carbohydrate is metered into the tank to maintain the culture as is known in the art. Samples are removed from the fermentor at regular intervals to measure enzyme titer using, for example, a colorimetric assay method. The fermentation process is halted when the enzyme production rate stops increasing according to the measurements.

An α-amylase variant secreted from the host cells may conveniently be recovered from the culture medium by well-known procedures, including separating the cells from the medium by centrifugation or filtration, and precipitating proteinaceous components of the medium by means of a salt such as ammonium sulfate, followed by the use of chromatographic procedures such as ion exchange chromatography, affinity chromatography, or the like.

Host cells may be cultured under suitable conditions which allow expression of the α-amylase variant proteins. Expression of the proteins may be constitutive such that they are continually produced, or inducible, requiring a stimulus to initiate expression. In the case of inducible expression, protein production can be initiated when required by addition of an inducer substance, e.g., dexamethasone, IPTG, or Sepharose, to the culture medium, for example. Polypeptides can also be produced recombinantly in an in vitro cell-free system, such as the TnT™ (Promega) rabbit reticulocyte system.

An α-amylase variant expressing host also can be cultured under aerobic conditions in the appropriate medium for the host. Shaking or a combination of agitation and aeration can be provided, with production occurring at the appropriate temperature for that host, e.g., from about 30° C. to about 75° C., depending on the needs of the host and production of the desired α-amylase variant. Culturing can occur from about 12 to about 100 hours or greater (and any hour value there between) or more particularly from 24 to 72 hours. Typically, the culture broth is at a pH of about 5.5 to about 8.0, again depending on the culture conditions needed for the host cell relative to production of the α-amylase variant.

### 4. PURIFICATION OF α-AMYLASE VARIANTS

Fermentation, separation, and concentration techniques are known in the art and conventional methods can be used in order to prepare the concentrated α-amylase variant containing solution. After fermentation, a fermentation broth is obtained, and the microbial cells and various suspended solids, including residual raw fermentation materials, are removed by conventional separation techniques to obtain an amylase solution. Filtration, centrifugation, microfiltration, rotary vacuum drum filtration, followed by ultra-filtration, extraction or chromatography, or the like are generally used.

It is desirable to concentrate the solution containing the α-amylase variant to optimize recovery, since the use of un-concentrated solutions requires increased incubation time to collect precipitates containing the purified α-amylase variant. The solution is concentrated using conventional techniques until the desired enzyme level is obtained. Concentration of the enzyme variant containing solution may be achieved by any of the techniques discussed above. In one embodiment, rotary vacuum evaporation and/or ultrafiltration is used. Alternatively, ultrafiltration can be used.

By “precipitation agent” for purposes of purification is meant a compound effective to precipitate the α-amylase variant from the concentrated enzyme variant solution in solid form, whatever its nature may be, i.e., crystalline, amorphous, or a blend of both. Precipitation can be performed using, for example, a metal halide precipitation agent. Metal halide precipitation agents include: alkali metal chlorides, alkali metal bromides and blends of two or more of these metal halides. The metal halide may be selected from the group consisting of sodium chloride, potassium chloride, sodium bromide, potassium bromide and blends of two or more of these metal halides. Suitable metal halides include sodium chloride and potassium chloride, particularly sodium chloride, which can further be used as a preservative.

The metal halide precipitation agent is used in an amount effective to precipitate the α-amylase variant. The selection of at least an effective amount and an optimum amount of metal halide effective to cause precipitation of the enzyme variant, as well as the conditions of the precipitation for maximum recovery including incubation time, pH, temperature and concentration of α-amylase variant, will be readily apparent to one of ordinary skill in the art after routine testing.

Generally, at least about 5% w/v (weight/volume) to about 25% w/v of metal halide is added to the concentrated enzyme variant solution, and usually at least 8% w/v. Generally, no more than about 25% w/v of metal halide is added to the concentrated enzyme variant solution and usually no more than about 20% w/v. The optimal concentration of the metal halide precipitation agent will depend, among others, on the nature of the specific α-amylase variant and on its concentration in the concentrated α-amylase variant solution.

Another alternative to effect precipitation of the enzyme is to use of organic compounds, which can be added to the concentrated enzyme variant solution. The organic compound precipitating agent can include: 4-hydroxybenzoic acid, alkali metal salts of 4-hydroxybenzoic acid, alkyl esters of 4-hydroxybenzoic acid, and blends of two or more of these organic compounds. The addition of the organic compound precipitation agents can take place prior to, simultaneously with or subsequent to the addition of the metal halide precipitation agent, and the addition of both precipitation agents, organic compound and metal halide, may be carried out sequentially or simultaneously. For a further description, see U.S. Pat. No. 5,281,526 to Genencor, for example.

Generally, the organic compound precipitation agents are selected from the group consisting of alkali metal salts of 4-hydroxybenzoic acid, such as sodium or potassium salts, and linear or branched alkyl esters of 4-hydroxybenzoic acid, wherein the alkyl group contains from 1 to 12 carbon atoms, and blends of two or more of these organic compounds. The organic compound precipitations agents can be for example linear or branched alkyl esters of 4-hydroxybenzoic acid, wherein the alkyl group contains from 1 to 10 carbon atoms, and blends of two or more of these organic compounds. Suitable organic compounds include linear alkyl esters of 4-hydroxybenzoic acid, wherein the alkyl group contains from 1 to 6 carbon atoms, and blends of two or more of these organic compounds. Methyl esters of 4-hydroxybenzoic acid, propyl ester of 4-hydroxybenzoic acid, butyl ester of 4-hydroxybenzoic acid, ethyl ester of 4-hydroxybenzoic acid and blends of two or more of these organic compounds can also be used. Additional organic compounds also include, but are not limited to, 4-hydroxybenzoic acid methyl ester (methyl PARABEN) and 4-hydroxybenzoic acid propyl ester (propyl PARABEN), which are also amylase preservative agents.

Addition of the organic compound precipitation agent provides the advantage of high flexibility of the precipitation conditions with respect to pH, temperature, α-amylase variant concentration, precipitation agent concentration, and time of incubation.

The organic compound precipitation agent is used in an amount effective to improve precipitation of the enzyme variant by means of the metal halide precipitation agent. The selection of at least an effective amount and an optimum amount of organic compound precipitation agent, as well as the conditions of the precipitation for maximum recovery including incubation time, pH, temperature and concentration of enzyme variant, will be readily apparent to one of ordinary skill in the art, in light of the present disclosure, after routine testing.

Generally, at least 0.01% w/v of organic compound precipitation agent is added to the concentrated enzyme variant solution and usually at least 0.02% w/v. Generally, no more than 0.3% w/v of organic compound precipitation agent is added to the concentrated enzyme variant solution and usually no more than 0.2% w/v.

The concentrated enzyme variant solution, containing the metal halide precipitation agent and, in one aspect, the organic compound precipitation agent, is adjusted to a pH that necessarily will depend on the enzyme variant to be purified. Generally, the pH is adjusted to a level near the isoelectric point (pI) of the amylase. For example, the pH can be adjusted within a range of about 2.5 pH units below the pI to about 2.5 pH units above the pI. For purposes of illustration, when the α-amylase variant is derived from B. licheniformis, the concentrated enzyme variant solution is usually adjusted to a pH of between about 5.5 and 9.7 and particularly to a pH of between about 6.5 and 9.0. The pH may be adjusted accordingly if the pI of the variant differs from the wild-type pI.

The incubation time necessary to obtain a purified enzyme variant precipitate depends on the nature of the specific enzyme variant, the concentration of enzyme, and the specific precipitation agent(s) and its (their) concentration. Generally, the time effective to precipitate the enzyme variant is between about 1 to about 30 hours; usually it does not exceed about 25 hours. In the presence of the organic compound precipitation agent, the time of incubation can still be reduced to less than about 10 hours, and in most cases even about 6 hours.

Generally, the temperature during incubation is between about 4° C. and about 50° C. Usually, the method is carried out at a temperature between about 10° C. and about 45° C., and particularly between about 20° C. and about 40° C. The optimal temperature for inducing precipitation varies according to the solution conditions and the enzyme variant or precipitation agent(s) used.

The overall recovery of purified enzyme variant precipitate, and the efficiency with which the process is conducted, is improved by agitating the solution comprising the enzyme variant, the added metal halide and the added organic compound. The agitation step is done both during addition of the metal halide and the organic compound, and during the subsequent incubation period. Suitable agitation methods include mechanical stirring or shaking, vigorous aeration, or any similar technique.

After the incubation period, the purified enzyme variant is then separated from the dissociated pigment and other impurities and collected by conventional separation techniques, such as filtration, centrifugation, microfiltration, rotary vacuum filtration, ultrafiltration, press filtration, cross membrane microfiltration, cross flow membrane microfiltration or the like. Cross membrane microfiltration can be one method used. Further purification of the purified enzyme variant precipitate can be obtained by washing the precipitate with water. For example, the purified enzyme variant precipitate is washed with water containing the metal halide precipitation agent, for example, with water containing the metal halide and the organic compound precipitation agents.

During the culturing, thermostable amylase extracellularly accumulates in the culture broth. For the isolation and purification of the desired α-amylase variant, the culture broth is centrifuged or filtered to eliminate cells, and the resulting cell-free liquid is used for the purification of the enzyme. In one embodiment, the cell-free broth is subjected to salting out using ammonium sulfate at about 70% saturation; the 70% saturation-precipitation fraction is then dissolved in a buffer and applied to a column such as a Sephadex G-100 column, and eluted to recover the enzyme variant active fraction. For further purification, a conventional procedure such as ion exchange chromatography may be used.

Purified enzyme variants are useful for all applications in which the enzyme variants are generally utilized. For example, they can be used in laundry detergents and spot removers, in the food industry, in starch processing and baking, and in pharmaceutical compositions as digestive aids. They can be made into a final product that is either liquid (solution, slurry) or solid (granular, powder).

Alternatively, the enzyme product can be recovered and a flocculating agent is added to the media in order to remove cells and cell debris by filtration or centrifugation without further purification of the enzyme.

The α-amylase variants produced and purified by the methods described above can be used in a variety of useful industrial applications. The variants possesses valuable properties facilitating applications related to fabric and household care (F&HC). For example, a variant can be used as a component in washing, dishwashing and hard-surface cleaning detergent compositions. Variants also are useful in the production of sweeteners and ethanol from starch, and/or for textile desizing. Variant α-amylases are particularly useful in starch-conversion processes, including starch liquefaction and/or saccharification processes, as described, for example, in WO 2005/111203 and U.S. Published Application No. 2006/0014265 (Genencor International, Inc.). These various uses of the α-amylase variants are described in more detail below.

### 5. CLEANING AND DISHWASHING COMPOSITIONS AND USE

The α-amylase variants discussed herein can be formulated in detergent compositions for use in cleaning dishes or other cleaning compositions, for example. These can be gels, powders or liquids. The compositions can comprise the α-amylase variant alone, other amylolytic enzymes, other cleaning enzymes, and other components common to cleaning compositions.

Thus, a dishwashing detergent composition can comprise a surfactant. The surfactant may be anionic, non-ionic, cationic, amphoteric or a mixture of these types. The detergent can contain 0% to about 90% by weight of a non-ionic surfactant, such as low- to non-foaming ethoxylated propoxylated straight-chain alcohols.

In the detergent applications, α-amylase variants are usually used in a liquid composition containing propylene glycol. The α-amylase variant is solubilized in for example in propylene glycol by circulating in a 25% volume/volume propylene glycol solution containing 10% calcium chloride.

The dishwashing detergent composition may contain detergent builder salts of inorganic and/or organic types. The detergent builders may be subdivided into phosphorus-containing and non-phosphorus-containing types. The detergent composition usually contains about 1% to about 90% of detergent builders. Examples of phosphorus-containing inorganic alkaline detergent builders, when present, include the water-soluble salts, especially alkali metal pyrophosphates, orthophosphates, and polyphosphates. An example of phosphorus-containing organic alkaline detergent builder, when present, includes the water-soluble salts of phosphonates. Examples of non-phosphorus-containing inorganic builders, when present, include water-soluble alkali metal carbonates, borates, and silicates, as well as the various types of water-insoluble crystalline or amorphous alumino silicates, of which zeolites are the best-known representatives.

Examples of suitable organic builders include the alkali metal; ammonium and substituted ammonium; citrates; succinates; malonates; fatty acid sulphonates; carboxymethoxy succinates; ammonium polyacetates; carboxylates; polycarboxylates; aminopolycarboxylates; polyacetyl carboxylates; and polyhydroxsulphonates.

Other suitable organic builders include the higher molecular weight polymers and co-polymers known to have builder properties, for example appropriate polyacrylic acid, polymaleic and polyacrylic/polymaleic acid copolymers, and their salts.

The cleaning composition may contain bleaching agents of the chlorine/bromine-type or the oxygen-type. Examples of inorganic chlorine/bromine-type bleaches are lithium, sodium or calcium hypochlorite, and hypobromite, as well as chlorinated trisodium phosphate. Examples of organic chlorine/bromine-type bleaches are heterocyclic N-bromo- and N-chloro-imides such as trichloroisocyanuric, tribromoisocyanuric, dibromoisocyanuric, and dichloroisocyanuric acids, and salts thereof with water-solubilizing cations such as potassium and sodium. Hydantoin compounds are also suitable.

The cleaning composition may contain oxygen bleaches, for example in the form of an inorganic persalt, optionally with a bleach precursor or as a peroxy acid compound. Typical examples of suitable peroxy bleach compounds are alkali metal perborates, both tetrahydrates and monohydrates, alkali metal percarbonates, persilicates, and perphosphates. Suitable activator materials include tetraacetylethylenediamine (TAED) and glycerol triacetate. Enzymatic bleach activation systems may also be present, such as perborate or percarbonate, glycerol triacetate and perhydrolase, as disclosed in WO 2005/056783.

The cleaning composition may be stabilized using conventional stabilizing agents for the enzyme(s), e.g., a polyol such as, e.g., propylene glycol, a sugar or a sugar alcohol, lactic acid, boric acid, or a boric acid derivative (e.g., an aromatic borate ester). The cleaning composition may also contain other conventional detergent ingredients, e.g., deflocculant material, filler material, foam depressors, anti-corrosion agents, soil-suspending agents, sequestering agents, anti-soil redeposition agents, dehydrating agents, dyes, bactericides, fluorescent agents, thickeners, and perfumes.

Finally, the α-amylase variants may be used in conventional dishwashing detergents, e.g., in any of the detergents described in the following patent publications, with the consideration that the α-amylase variants disclosed herein are used instead of, or in addition to, any α-amylase disclosed in the listed patents and published applications: CA 2006687, GB 2200132, GB 2234980, GB 2228945, DE 3741617, DE 3727911, DE 4212166, DE 4137470, DE 3833047, DE 4205071, WO 93/25651, WO 93/18129, WO 93/04153, WO 92/06157, WO 92/08777, WO 93/21299, WO 93/17089, WO 93/03129, EP 481547, EP 530870, EP 533239, EP 554943, EP 429124, EP 346137, EP 561452, EP 318204, EP 318279, EP 271155, EP 271156, EP 346136, EP 518719, EP 518720, EP 518721, EP 516553, EP 561446, EP 516554, EP 516555, EP 530635, EP 414197, and U.S. Pat. Nos. 5,112,518; 5,141,664; and 5,240,632.

### 6. LAUNDRY DETERGENT COMPOSITIONS AND USE

According to the embodiment, one or more α-amylase variants may typically be a component of a detergent composition. As such, it may be included in the detergent composition in the form of a non-dusting granulate, a stabilized liquid, or a protected enzyme. Non-dusting granulates may be produced, e.g., as disclosed in U.S. Pat. Nos. 4,106,991 and 4,661,452 and may optionally be coated by methods known in the art. Examples of waxy coating materials are poly(ethylene oxide) products; (polyethyleneglycol, PEG) with mean molar weights of 1,000 to 20,000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in, for example, GB Patent No. 1483591. Liquid enzyme preparations may, for instance, be stabilized by adding a polyol such as propylene glycol, a sugar or sugar alcohol, lactic acid or boric acid according to established methods. Other enzyme stabilizers are well known in the art. Protected enzymes may be prepared according to the method disclosed in U.S. Pat. No. 5,879,920 (Genencor Int'l, Inc.) or EP 238216, for example. Polyols have long been recognized as stabilizers of proteins as well as for improving the solubility of proteins. See, e.g., Kaushik et al., “Why is trehalose an exceptional protein stabilizer? An analysis of the thermal stability of proteins in the presence of the compatible osmolyte trehalose” J. Biol. Chem. 278: 26458-65 (2003) and references cited therein; and M. Conti et al., “Capillary isoelectric focusing: the problem of protein solubility,” J. Chromatography 757: 237-245 (1997).

The detergent composition may be in any convenient form, e.g., as gels, powders, granules, pastes, or liquids. A liquid detergent may be aqueous, typically containing up to about 70% of water, and 0% to about 30% of organic solvent, it may also be in the form of a compact gel type containing only about 30% water.

The detergent composition comprises one or more surfactants, each of which may be anionic, nonionic, cationic, or zwitterionic. The detergent will usually contain 0% to about 50% of anionic surfactant, such as linear alkylbenzenesulfonate (LAS); α-olefinsulfonate (AOS); alkyl sulfate (fatty alcohol sulfate) (AS); alcohol ethoxysulfate (AEOS or AES); secondary alkanesulfonates (SAS); α-sulfo fatty acid methyl esters; alkyl- or alkenylsuccinic acid; or soap. The composition may also contain 0% to about 40% of nonionic surfactant such as alcohol ethoxylate (AEO or AE), carboxylated alcohol ethoxylates, nonylphenol ethoxylate, alkylpolyglycoside, alkyldimethylamineoxide, ethoxylated fatty acid monoethanolamide, fatty acid monoethanolamide, or polyhydroxy alkyl fatty acid amide, as described in WO 92/06154, for example.

The detergent composition may additionally comprise one or more other enzymes, such as lipase, cutinase, protease, cellulase, peroxidase, and/or laccase in any combination.

The detergent may contain about 1% to about 65% of a detergent builder or complexing agent such as zeolite, diphosphate, triphosphate, phosphonate, citrate, nitrilotriacetic acid (NTA), ethylenediaminetetraacetic acid (EDTA), diethylenetriaminepentaacetic acid (DTMPA), alkyl- or alkenylsuccinic acid, soluble silicates or layered silicates (e.g., SKS-6 from Hoechst). The detergent may also be unbuilt, i.e., essentially free of detergent builder. Enzymes may be used in any composition compatible with the stability of the enzyme. Enzymes can be protected against generally deleterious components by known forms of encapsulation, as by granulation or sequestration in hydro gels, for example. Enzymes and specifically α-amylases either with or without the starch binding domains are not limited to laundry and dishwashing applications, but may find use in surface cleaners and ethanol production from starch or biomass.

The detergent may comprise one or more polymers. Examples include carboxymethylcellulose (CMC), poly(vinylpyrrolidone) (PVP), polyethyleneglycol (PEG), poly(vinyl alcohol) (PVA), polycarboxylates such as polyacrylates, maleic/acrylic acid copolymers and lauryl methacrylate/acrylic acid copolymers.

The detergent may contain a bleaching system, which may comprise a H₂O₂ source such as perborate or percarbonate optionally combined with a peracid-forming bleach activator, such as TAED or nonanoyloxybenzenesulfonate (NOBS). Alternatively, the bleaching system may comprise peroxy acids of the amide, imide, or sulfone type, for example. The bleaching system can also be an enzymatic bleaching system where a perhydrolase activates peroxide, such as that described in WO 2005/056783.

The enzymes of the detergent composition may be stabilized using conventional stabilizing agents, e.g., a polyol such as propylene glycol or glycerol; a sugar or sugar alcohol; lactic acid; boric acid or a boric acid derivative, such as an aromatic borate ester; and the composition may be formulated as described in WO 92/19709 and WO 92/19708, for example.

The detergent may also contain other conventional detergent ingredients such as fabric conditioners including clays, foam boosters, suds suppressors, anti-corrosion agents, soil-suspending agents, anti-soil redeposition agents, dyes, bactericides, optical brighteners, or perfume, for example. The pH (measured in aqueous solution at use concentration) is usually neutral or alkaline, e.g., pH about 7.0 to about 11.0.

The α-amylase variant may be incorporated in concentrations conventionally employed in detergents. It is at present contemplated that, in the detergent composition, the α-amylase variant may be added in an amount corresponding to 0.00001-1.0 mg (calculated as pure enzyme protein) of α-amylase variant per liter of wash liquor. Particular forms of detergent compositions comprising the α-amylase variants can be formulated to include:

(1) A detergent composition formulated as a granulate having a bulk density of at least 600 g/L comprising linear alkylbenzenesulfonate (calculated as acid) about 7% to about 12%; alcohol ethoxysulfate (e.g., C₁₂₋₁₈ alcohol, 1-2 ethylene oxide (EO)) or alkyl sulfate (e.g., C₁₆₋₁₈) about 1% to about 4%; alcohol ethoxylate (e.g., C₁₄₋₁₅ alcohol, 7 EO) about 5% to about 9%; sodium carbonate (e.g., Na₂CO₃) about 14% to about 20%; soluble silicate about 2 to about 6%; zeolite (e.g., NaAlSiO₄) about 15% to about 22%; sodium sulfate (e.g., Na₂SO₄) 0% to about 6%; sodium citrate/citric acid (e.g., C₆H₅Na₃O₇/C₆H₈O₇) about 0% to about 15%; sodium perborate (e.g., NaBO₃H₂O) about 11% to about 18%; TAED about 2% to about 6%; carboxymethylcellulose (CMC) and 0% to about 2%; polymers (e.g., maleic/acrylic acid, copolymer, PVP, PEG) 0-3%; enzymes (calculated as pure enzyme) 0.0001-0.1% protein; and minor ingredients (e.g., suds suppressors, perfumes, optical brightener, photobleach) 0-5%.

(2) A detergent composition formulated as a granulate having a bulk density of at least 600 g/L comprising linear alkylbenzenesulfonate (calculated as acid) about 6% to about 11%; alcohol ethoxysulfate (e.g., C₁₂₋₁₈ alcohol, 1-2 EO) or alkyl sulfate (e.g., C₁₆₋₁₈) about 1% to about 3%; alcohol ethoxylate (e.g., C₁₄₋₁₅ alcohol, 7 EU) about 5% to about 9%; sodium carbonate (e.g., Na₂CO₃) about 15% to about 21%; soluble silicate about 1% to about 4%; zeolite (e.g., NaAlSiO₄) about 24% to about 34%; sodium sulfate (e.g., Na₂SO₄) about 4% to about 10%; sodium citrate/citric acid (e.g., C₆H₅Na₃O₇/C₆H₈O₇) 0% to about 15%; carboxymethylcellulose (CMC) 0% to about 2%; polymers (e.g., maleic/acrylic acid copolymer, PVP, PEG) 1-6%; enzymes (calculated as pure enzyme protein) 0.0001-0.1%; minor ingredients (e.g., suds suppressors, perfume) 0-5%.

(3) A detergent composition formulated as a granulate having a bulk density of at least 600 g/L comprising linear alkylbenzenesulfonate (calculated as acid) about 5% to about 9%; alcohol ethoxylate (e.g., C₁₂₋₁₅ alcohol, 7 EU) about 7% to about 14%; Soap as fatty acid (e.g., C₁₆₋₂₂ fatty acid) about 1 to about 3%; sodium carbonate (as Na₂CO₃) about 10% to about 17%; soluble silicate about 3% to about 9%; zeolite (as NaAlSiO₄) about 23% to about 33%; sodium sulfate (e.g., Na₂SO₄) 0% to about 4%; sodium perborate (e.g., NaBO₃H₂O) about 8% to about 16%; TAED about 2% to about 8%; phosphonate (e.g., EDTMPA) 0% to about 1%; carboxymethylcellulose (CMC) 0% to about 2%; polymers (e.g., maleic/acrylic acid copolymer, PVP, PEG) 0-3%; enzymes (calculated as pure enzyme protein) 0.0001-0.1%; minor ingredients (e.g., suds suppressors, perfume, optical brightener) 0-5%.

(4) A detergent composition formulated as a granulate having a bulk density of at least 600 g/L comprising linear alkylbenzenesulfonate (calculated as acid) about 8% to about 12%; alcohol ethoxylate (e.g., C₁₂₋₁₅ alcohol, 7 EO) about 10% to about 25%; sodium carbonate (as Na₂CO₃) about 14% to about 22%; soluble silicate about 1% to about 5%; zeolite (e.g., NaAlSiO₄) about 25% to about 35%; sodium sulfate (e.g., Na₂SO₄) 0% to about 10%; carboxymethylcellulose (CMC) 0% to about 2%; polymers (e.g., maleic/acrylic acid copolymer, PVP, PEG) 1-3%; enzymes (calculated as pure enzyme protein) 0.0001-0.1%; and minor ingredients (e.g., suds suppressors, perfume) 0-5%.

(5) An aqueous liquid detergent composition comprising linear alkylbenzenesulfonate (calculated as acid) about 15% to about 21%; alcohol ethoxylate (e.g., C₁₂₋₁₅ alcohol, 7 EO or C₁₂₋₁₅ alcohol, 5 EO) about 12% to about 18%; soap as fatty acid (e.g., oleic acid) about 3% to about 13%; alkenylsuccinic acid (C₁₂₋₁₄) 0% to about 13%; aminoethanol about 8% to about 18%; citric acid about 2% to about 8%; phosphonate 0% to about 3%; polymers (e.g., PVP, PEG) 0% to about 3%; borate (e.g., B₄O₇) 0% to about 2%; ethanol 0% to about 3%; propylene glycol about 8% to about 14%; enzymes (calculated as pure enzyme protein) 0.0001-0.1%; and minor ingredients (e.g., dispersants, suds suppressors, perfume, optical brightener) 0-5%.

(6) An aqueous structured liquid detergent composition comprising linear alkylbenzenesulfonate (calculated as acid) about 15% to about 21%; alcohol ethoxylate (e.g., C₁₂₋₁₅ alcohol, 7 EO, or C₁₂₋₁₅ alcohol, 5 EO) 3-9%; soap as fatty acid (e.g., oleic acid) about 3% to about 10%; zeolite (as NaAlSiO₄) about 14% to about 22%; potassium citrate about 9% to about 18%; borate (e.g., B₄O₇) 0% to about 2%; carboxymethylcellulose (CMC) 0% to about 2%; polymers (e.g., PEG, PVP) 0% to about 3%; anchoring polymers (e.g., lauryl methacrylate/acrylic acid copolymer); molar ratio 25:1, MW 3800) 0% to about 3%; glycerol 0% to about 5%; enzymes (calculated as pure enzyme protein) 0.0001-0.1%; and minor ingredients (e.g., dispersants, suds suppressors, perfume, optical brighteners) 0-5%.

(7) A detergent composition formulated as a granulate having a bulk density of at least 600 g/L comprising fatty alcohol sulfate about 5% to about 10%; ethoxylated fatty acid monoethanolamide about 3% to about 9%; soap as fatty acid 0-3%; sodium carbonate (e.g., Na₂CO₃) about 5% to about 10%; Soluble silicate about 1% to about 4%; zeolite (e.g., NaAlSiO₄) about 20% to about 40%; sodium sulfate (e.g., Na₂SO₄) about 2% to about 8%; sodium perborate (e.g., NaBO₃H₂O) about 12% to about 18%; TAED about 2% to about 7%; polymers (e.g., maleic/acrylic acid copolymer, PEG) about 1% to about 5%; enzymes (calculated as pure enzyme protein) 0.0001-0.1%; and minor ingredients (e.g., optical brightener, suds suppressors, perfume) 0-5%.

(8) A detergent composition formulated as a granulate comprising linear alkylbenzenesulfonate (calculated as acid) about 8% to about 14%; ethoxylated fatty acid monoethanolamide about 5% to about 11%; soap as fatty acid 0% to about 3%; sodium carbonate (e.g., Na₂CO₃) about 4% to about 10%; soluble silicate about 1% to about 4%; zeolite (e.g., NaAlSiO₄) about 30% to about 50%; sodium sulfate (e.g., Na₂SO₄) about 3% to about 11%; sodium citrate (e.g., C₆H₅Na₃O₇) about 5% to about 12%; polymers (e.g., PVP, maleic/acrylic acid copolymer, PEG) about 1% to about 5%; enzymes (calculated as pure enzyme protein) 0.0001-0.1%; and minor ingredients (e.g., suds suppressors, perfume) 0-5%.

(9) A detergent composition formulated as a granulate comprising linear alkylbenzenesulfonate (calculated as acid) about 6% to about 12%; nonionic surfactant about 1% to about 4%; soap as fatty acid about 2% to about 6%; sodium carbonate (e.g., Na₂CO₃) about 14% to about 22%; zeolite (e.g., NaAlSiO₄) about 18% to about 32%; sodium sulfate (e.g., Na₂SO₄) about 5% to about 20%; sodium citrate (e.g., C₆H₅Na₃O₇) about 3% to about 8%; sodium perborate (e.g., NaBO₃H₂O) about 4% to about 9%; bleach activator (e.g., NOBS or TAED) about 1% to about 5%; carboxymethylcellulose (CMC) 0% to about 2%; polymers (e.g., polycarboxylate or PEG) about 1% to about 5%; enzymes (calculated as pure enzyme protein) 0.0001-0.1%; and minor ingredients (e.g., optical brightener, perfume) 0-5%.

(10) An aqueous liquid detergent composition comprising linear alkylbenzenesulfonate (calculated as acid) about 15% to about 23%; alcohol ethoxysulfate (e.g., C₁₂₋₁₅ alcohol, 2-3 EO) about 8% to about 15%; alcohol ethoxylate (e.g., C₁₂₋₁₅ alcohol, 7 EO, or C₁₂₋₁₅ alcohol, 5 EO) about 3% to about 9%; soap as fatty acid (e.g., lauric acid) 0% to about 3%; aminoethanol about 1% to about 5%; sodium citrate about 5% to about 10%; hydrotrope (e.g., sodium toluensulfonate) about 2% to about 6%; borate (e.g., B₄O₇) 0% to about 2%; carboxymethylcellulose 0% to about 1%; ethanol about 1% to about 3%; propylene glycol about 2% to about 5%; enzymes (calculated as pure enzyme protein) 0.0001-0.1%; and minor ingredients (e.g., polymers, dispersants, perfume, optical brighteners) 0-5%.

(11) An aqueous liquid detergent composition comprising linear alkylbenzenesulfonate (calculated as acid) about 20% to about 32%; alcohol ethoxylate (e.g., C₁₂₋₁₅ alcohol, 7 EO, or C₁₂₋₁₅ alcohol, 5 EO) 6-12%; aminoethanol about 2% to about 6%; citric acid about 8% to about 14%; borate (e.g., B₄O₇) about 1% to about 3%; polymer (e.g., maleic/acrylic acid copolymer, anchoring polymer, such as lauryl methacrylate/acrylic acid copolymer) 0% to about 3%; glycerol about 3% to about 8%; enzymes (calculated as pure enzyme protein) 0.0001-0.1%; and minor ingredients (e.g., hydrotropes, dispersants, perfume, optical brighteners) 0-5%.

(12) A detergent composition formulated as a granulate having a bulk density of at least 600 g/L comprising anionic surfactant (linear alkylbenzenesulfonate, alkyl sulfate, α-olefinsulfonate, α-sulfo fatty acid methyl esters, alkanesulfonates, soap) about 25% to about 40%; nonionic surfactant (e.g., alcohol ethoxylate) about 1% to about 10%; sodium carbonate (e.g., Na₂CO₃) about 8% to about 25%; soluble silicates about 5% to about 15%; sodium sulfate (e.g., Na₂SO₄) 0% to about 5%; zeolite (NaAlSiO₄) about 15% to about 28%; sodium perborate (e.g., NaBO₃.4H₂O) 0% to about 20%; bleach activator (TAED or NOBS) about 0% to about 5%; enzymes (calculated as pure enzyme protein) 0.0001-0.1%; minor ingredients (e.g., perfume, optical brighteners) 0-3%.

(13) Detergent compositions as described in compositions 1)-12) supra, wherein all or part of the linear alkylbenzenesulfonate is replaced by (C₁₂-C₁₈) alkyl sulfate.

(14) A detergent composition formulated as a granulate having a bulk density of at least 600 g/L comprising (C₁₂-C₁₈) alkyl sulfate about 9% to about 15%; alcohol ethoxylate about 3% to about 6%; polyhydroxy alkyl fatty acid amide about 1% to about 5%; zeolite (e.g., NaAlSiO₄) about 10% to about 20%; layered disilicate (e.g., SK56 from Hoechst) about 10% to about 20%; sodium carbonate (e.g., Na₂CO₃) about 3% to about 12%; soluble silicate 0% to about 6%; sodium citrate about 4% to about 8%; sodium percarbonate about 13% to about 22%; TAED about 3% to about 8%; polymers (e.g., polycarboxylates and PVP) 0% to about 5%; enzymes (calculated as pure enzyme protein) 0.0001-0.1%; and minor ingredients (e.g., optical brightener, photobleach, perfume, suds suppressors) 0-5%.

(15) A detergent composition formulated as a granulate having a bulk density of at least 600 g/L comprising (C₁₂-C₁₈) alkyl sulfate about 4% to about 8%; alcohol ethoxylate about 11% to about 15%; soap about 1% to about 4%; zeolite MAP or zeolite A about 35% to about 45%; sodium carbonate (as Na₂CO₃) about 2% to about 8%; soluble silicate 0% to about 4%; sodium percarbonate about 13% to about 22%; TAED 1-8%; carboxymethylcellulose (CMC) 0% to about 3%; polymers (e.g., polycarboxylates and PVP) 0% to about 3%; enzymes (calculated as pure enzyme protein) 0.0001-0.1%; and minor ingredients (e.g., optical brightener, phosphonate, perfume) 0-3%.

(16) Detergent formulations as described in 1)-15) supra, which contain a stabilized or encapsulated peracid, either as an additional component or as a substitute for already specified bleach systems.

(17) Detergent compositions as described supra in 1), 3), 7), 9), and 12), wherein perborate is replaced by percarbonate.

(18) Detergent compositions as described supra in 1), 3), 7), 9), 12), 14), and 15), which additionally contains a manganese catalyst.

(19) Detergent composition formulated as a non-aqueous detergent liquid comprising a liquid nonionic surfactant such as, e.g., linear alkoxylated primary alcohol, a builder system (e.g., phosphate), an enzyme(s), and alkali. The detergent may also comprise anionic surfactant and/or a bleach system.

In another embodiment, the 2,6-β-D-fructan hydrolase can be incorporated in detergent compositions and used for removal/cleaning of biofilm present on household and/or industrial textile/laundry.

The detergent composition may for example be formulated as a hand or machine laundry detergent composition, including a laundry additive composition suitable for pre-treatment of stained fabrics and a rinse added fabric softener composition, or be formulated as a detergent composition for use in general household hard surface cleaning operations, or be formulated for hand or machine dishwashing operations.

In a specific aspect, the detergent composition can comprise 2,6-β-D-fructan hydrolase, one or more α-amylase variants, and one or more other cleaning enzymes, such as a protease, a lipase, a cutinase, a carbohydrase, a cellulase, a pectinase, a mannanase, an arabinase, a galactanase, a xylanase, an oxidase, a laccase, and/or a peroxidase, and/or combinations thereof.

In general the properties of the chosen enzyme(s) should be compatible with the selected detergent, (e.g., pH-optimum, compatibility with other enzymatic and non-enzymatic ingredients, etc.), and the enzyme(s) should be present in effective amounts.

Proteases: suitable proteases include those of animal, vegetable or microbial origin. Chemically modified or protein engineered mutants are also suitable. The protease may be a serine protease or a metalloprotease, e.g., an alkaline microbial protease or a trypsin-like protease. Examples of alkaline proteases are subtilisins, especially those derived from Bacillus sp., e.g., subtilisin Novo, subtilisin Carlsberg, subtilisin 309 (see, e.g., U.S. Pat. No. 6,287,841), subtilisin 147, and subtilisin 168 (see, e.g., WO 89/06279). Examples of trypsin-like proteases are trypsin (e.g., of porcine or bovine origin), and Fusarium proteases (see, e.g., WO 89/06270 and WO 94/25583). Examples of useful proteases also include but are not limited to the variants described in WO 92/19729 and WO 98/20115. Suitable commercially available protease enzymes include Alcalase®, Savinase®, Esperase®, and Kannase™ (Novozymes, formerly Novo Nordisk A/S); Maxatase®, Maxacal™, Maxapem™, Properase™, Purafect®, Purafect OxP™, FN2™, and FN3™ (Danisco US Inc., Genencor Division; previously known as Genencor International, Inc.).

Lipases: suitable lipases include those of bacterial or fungal origin. Chemically modified or protein engineered mutants are included. Examples of useful lipases include, but are not limited to, lipases from Humicola (synonym Thermomyces), e.g. H. lanuginosa (T. lanuginosus) (see, e.g., EP 258068 and EP 305216) and H. insolens (see, e.g., WO 96/13580); a Pseudomonas lipase (e.g., from P. alcaligenes or P. pseudoalcaligenes; see, e.g., EP 218 272), P. cepacia (see, e.g., EP 331 376), P. stutzeri (see, e.g., GB 1,372,034), P. fluorescens, Pseudomonas sp. strain SD 705 (see, e.g., WO 95/06720 and WO 96/27002), P. wisconsinensis (see, e.g., WO 96/12012); a Bacillus lipase (e.g., from B. subtilis; see, e.g., Dartois et al. Biochemica Biophysica Acta, 1131: 253-360 (1993)), B. stearothermophilus (see, e.g., JP 64/744992), or B. pumilus (see, e.g., WO 91/16422). Additional lipase variants contemplated for use in the formulations include those described, for example, in: WO 92/05249, WO 94/01541, WO 95/35381, WO 96/00292, WO 95/30744, WO 94/25578, WO 95/14783, WO 95/22615, WO 97/04079, WO 97/07202, EP 407225, and EP 260105. Some commercially available lipase enzymes include Lipolase® and Lipolase® Ultra (Novozymes, formerly Novo Nordisk A/S).

Polyesterases: Suitable polyesterases include, but are not limited to, those described in WO 01/34899 (Genencor International, Inc.) and WO 01/14629 (Genencor Interntional, Inc.), and can be included in any combination with other enzymes discussed herein.

Amylases: The compositions can be combined with other α-amylases, such as a non-variant α-amylase. These can include commercially available amylases, such as but not limited to Duramyl®, Termamyl™, Fungamyl® and BAN™ (Novozymes, formerly Novo Nordisk A/S), Rapidase®, and Purastar® (Danisco US Inc., Genencor Division; formerly Genencor International, Inc.).

Cellulases: Cellulases can be added to the compositions. Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307; 5,648,263; 5,691,178; 5,776,757; and WO 89/09259, for example. Exemplary cellulases contemplated for use are those having color care benefit for the textile. Examples of such cellulases are cellulases described in EP 0495257; EP 531 372; WO 99/25846 (Genencor International, Inc.), WO 96/34108 (Genencor International, Inc.), WO 96/11262; WO 96/29397; and WO 98/08940, for example. Other examples are cellulase variants, such as those described in WO 94/07998; WO 98/12307; WO 95/24471; PCT/DK98/00299; EP 531315 (Novo Nordisk); U.S. Pat. Nos. 5,457,046; 5,686,593; and 5,763,254. Commercially available cellulases include Celluzyme® and Carezyme® (Novozymes, formerly Novo Nordisk A/S); Clazinase™ and Puradax® HA (Danisco US Inc., Genencor Division; previously known as Genencor International, Inc.); and KAC-500(B)™ (Kao Corporation).

Peroxidases/Oxidases: Suitable peroxidases/oxidases contemplated for use in the compositions include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257.

The detergent enzyme(s) may be included in a detergent composition by adding separate additives containing one or more enzymes, or by adding a combined additive comprising all of these enzymes. A detergent additive, i.e., a separate additive or a combined additive, can be formulated as a granulate, liquid, slurry, etc. Suitable granulate detergent additive formulations include non-dusting granulates.

Non-dusting granulates may be produced, e.g., as disclosed in U.S. Pat. Nos. 4,106,991 and 4,661,452 and optionally may be coated by methods known in the art. Examples of waxy coating materials are poly(ethylene oxide) products (e.g., polyethyleneglycol, PEG) with mean molar weights of 1,000 to 20,000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591, for example. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238216.

The detergent composition may be in any convenient form, e.g., a bar, tablet, gel, powder, granule, paste, or liquid. A liquid detergent may be aqueous, typically containing up to about 70% water, and 0% to about 30% organic solvent. Compact detergent gels containing 30% or less water are also contemplated. The detergent composition comprises one or more surfactants, which may be non-ionic, including semi-polar, anionic, cationic, or zwitterionic, or any combination thereof. The surfactants are typically present at a level of from 0.1% to 60% by weight.

When included therein the detergent typically will contain from about 1% to about 40% of an anionic surfactant, such as linear alkylbenzenesulfonate, α-olefinsulfonate, alkyl sulfate (fatty alcohol sulfate), alcohol ethoxysulfate, secondary alkanesulfonate, α-sulfo fatty acid methyl ester, alkyl- or alkenylsuccinic acid, or soap.

When included therein, the detergent will usually contain from about 0.2% to about 40% of a non-ionic surfactant such as alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl-N-alkyl derivatives of glucosamine (“glucamides”).

The detergent may contain 0% to about 65% of a detergent builder or complexing agent such as zeolite, diphosphate, triphosphate, phosphonate, carbonate, citrate, nitrilotriacetic acid, ethylenediaminetetraacetic acid (EDTA), diethylenetriaminepentaacetic acid, alkyl- or alkenylsuccinic acid, soluble silicates or layered silicates (e.g., SKS-6 from Hoechst).

The detergent may comprise one or more polymers. Examples are carboxymethylcellulose (CMC), poly(vinylpyrrolidone) (PVP), poly(ethylene glycol) (PEG), poly(vinyl alcohol) (PVA), poly(vinylpyridine-N-oxide), poly(vinylimidazole), polycarboxylates, e.g., polyacrylates, maleic/acrylic acid copolymers), and lauryl methacrylate/acrylic acid copolymers.

The detergent may contain a bleaching system that may comprise a source of H₂O₂, such as perborate or percarbonate, which may be combined with a peracid-forming bleach activator (e.g., tetraacetylethylenediamine or nonanoyloxybenzenesulfonate). Alternatively, the bleaching system may comprise peroxyacids (e.g., the amide-, imide-, or sulfone-type peroxyacids). The bleaching system can also be an enzymatic bleaching system.

The enzyme(s) of the detergent composition may be stabilized using conventional stabilizing agents, e.g., polyol (e.g., propylene glycol or glycerol), a sugar or sugar alcohol, lactic acid, boric acid, a boric acid derivative (e.g., an aromatic borate ester), or a phenyl boronic acid derivative (e.g., 4-formylphenyl boronic acid). The composition may be formulated as described in WO 92/19709 and WO 92/19708.

The detergent may also contain other conventional detergent ingredients such as e.g., fabric conditioners including clays, foam boosters, suds suppressors, anti-corrosion agents, soil-suspending agents, anti-soil redeposition agents, dyes, bactericides, optical brighteners, hydrotropes, tarnish inhibitors, or perfumes.

It is contemplated that in the detergent compositions, the enzyme variants may be added in an amount corresponding to about 0.01 to about 100 mg of enzyme protein per liter of wash liquor, particularly about 0.05 to about 5.0 mg of enzyme protein per liter of wash liquor, or even more particularly in 0.1 to about 1.0 mg of enzyme protein per liter of wash liquor.

### 6.1. Methods of Assessing Detergent Compositions

Numerous α-amylase cleaning assays exist. Exemplary description of testing cleaning includes the following. A “swatch” is a piece of material such as a fabric that has a stain applied thereto. The material can be, for example, fabrics made of cotton, polyester or mixtures of natural and synthetic fibers. Alternatively, the material can be paper, such as filter paper or nitrocellulose, or a piece of a hard material, such as ceramic, metal, or glass. For α-amylases, the stain is starch based, but can include blood, milk, ink, grass, tea, wine, spinach, gravy, chocolate egg, cheese, clay, pigment, oil, or mixtures of these compounds. In one embodiment, the α-amlyase variant is tested in a BMI (blood/milk/ink) assay.

A “smaller swatch” is a piece of the swatch that has been cut with a single hole punch device, for example, or a custom manufactured 96-hole punch device, where the pattern of the multi-hole punch is matched to standard 96-well microtiter plates, or has been otherwise removed from the swatch. The swatch can be of textile, paper, metal, or other suitable material. The smaller swatch can have the stain affixed either before or after it is placed into the well of a 24-, 48- or 96-well microtiter plate. The smaller swatch also can be made by applying a stain to a small piece of material. For example, the smaller swatch can be a piece of fabric with a stain ⅝″ or 0.25″ in diameter. The custom manufactured punch is designed in such a manner that it delivers 96 swatches simultaneously to all wells of a 96-well plate. The device allows delivery of more than one swatch per well by simply loading the same 96-well plate multiple times. Multi-hole punch devices can be conceived to deliver simultaneously swatches to any format plate, including, but not limited to, 24-well, 48-well, and 96-well plates. In another conceivable method, the soiled test platform can be a bead made of either metal, plastic, glass, ceramic, or other suitable material that is coated with the soil substrate. The one or more coated beads are then placed into wells of 96-, 48-, or 24-well plates or larger formats, containing suitable buffer and enzyme. In this case, supernatant can be examined for released soil either by direct absorbance measurement or after a secondary color development reaction. Analysis of the released soil might also be taken by mass spectral analysis.

In one embodiment, a treatment protocol provides control over degree of fixation of a stain. As a result, it is possible to produce swatches that, for example, release varying amounts of stain when washed in the absence of the enzyme being tested. The use of fixed swatches leads to a dramatic improvement of the signal-to-noise ratio in the wash assays. Furthermore, by varying the degree of fixation, one can generate stains that give optimum results under the various cleaning conditions.

Swatches having stains of known “strength” on various types of material are commercially available (EMPA, St. Gallen, Switzerland; Testgewebe GmbH, Krefeld Germany; or Center for Test Materials, Vlaardingen, The Netherlands) and/or can be made by the practitioner (Morris and Prato, Textile Research Journal 52(4): 280-286 (1982)). Swatches can comprise, for example, a cotton-containing fabric containing a stain made by blood/milk/ink (BMI), spinach, grass, or chocolate/milk/soot. A BMI stain can be fixed to cotton with 0.0003% to 0.3% hydrogen peroxide, for example. Other combinations include grass or spinach fixed with 0.001% to 1% glutaraldehyde, gelatin and Coomassie stain fixed with 0.001% to 1% glutaraldehyde, or chocolate, milk and soot fixed with 0.001% to 1% glutaraldehyde.

The swatch can also be agitated during incubation with the enzyme and/or detergent formulation. Wash performance data is dependent on the orientation of the swatches in the wells (horizontal versus vertical), particularly in the 96-well plate. This would indicate that mixing was insufficient during the incubation period. Although there are a number of ways to ensure sufficient agitation during incubation, a plate holder in which the microtiter plate is sandwiched between two plates of aluminum can be constructed. This can be as simple as placing, for example, an adhesive plate sealer over the wells then clamping the two aluminum plates to the 96-well plate with any type of appropriate, commercially available clamps. It can then be mounted in a commercial incubator shaker. Setting the shaker to about 400 rpm results in very efficient mixing, while leakage or cross-contamination is efficiently prevented by the holder.

Trinitrobenzenesulfonic acid (TNBS) can be used to quantify the concentration of amino groups in the wash liquor. This can serve as a measure of the amount of protein that was removed from the swatch (see, e.g., Cayot and Tainturier, Anal. Biochem. 249: 184-200 (1997)). However, if a detergent or an enzyme sample leads to the formation of unusually small peptide fragments (for example, from the presence of peptidases in the sample), then one will obtain a larger TNBS signal, i.e., more “noise.”

Another means of measuring wash performance of blood/milk/ink that is based on ink release that can be quantified by measuring the absorbance of the wash liquor. The absorbance can be measured at any wavelength between 350 and 800 nm. In one embodiment, the wavelength is measured at 410 nm or 620 nm. The wash liquor can also be examined to determine the wash performance on stains containing grass, spinach, gelatin or Coomassie stain. Suitable wavelengths for these stains include and 670 nm for spinach or grass and 620 nm for gelatin or Coomassie. For example, an aliquot of the wash liquor (typically 100-150 μl. from a 96-well microplate, for example) is removed and placed in a cuvette or multiwell microplate. This is then placed in a spectrophotometer and the absorbance is read at an appropriate wavelength. The system also can be used to determine a suitable enzyme and/or detergent composition for dish washing, for example, using a blood/milk/ink stain on a suitable substrate, such as cloth, plastic or ceramic.

In one aspect, the a BMI stain is fixed to cotton by applying 0.3% hydrogen peroxide to the BMI/cotton swatch for 30 minutes at 25° C. or by applying 0.03% hydrogen peroxide to the BMI/cotton swatch for 30 minutes at 60° C. Smaller swatches of approximately 0.25″ are cut from the BMI/cotton swatch and placed in the wells of a 96-well microtiter plate. Into each well, a known mixture of a detergent composition and an enzyme such as a variant protein is placed. After placing an adhesive plate sealer onto the top of the microtiter plate, the microtiter plate is clamped to an aluminum plate and agitated on an orbital shaker at approximately 250 rpm for about 10 to 60 minutes. At the end of this time, the supernatants are transferred to wells in a new microtiter plate and the absorbance of the ink at 620 nm is measured. This can be similarly tests with spinach stains or grass stains fixed to cotton by applying 0.01% glutaraldehyde to the spinach/cotton swatch or grass/cotton swatch for 30 minutes at 25° C. The same can be done with chocolate, milk, and/or soot stains.

### 7. BIOFILM REMOVAL COMPOSITIONS AND USE

The composition may comprise one α-amylase variants as the major enzymatic component, e.g., a mono-component composition for use in removing biofilms. Alternatively, the composition may comprise multiple enzymatic activities, such as multiple amylases, or a cocktail of enzymes including an aminopeptidase, amylase (β- or α- or gluco-amylase), carbohydrase, carboxypeptidase, catalase, cellulase, chitinase, cutinase, cyclodextrin glycosyltransferase, deoxyribonuclease, esterase, α-galactosidase, β-galactosidase, glucoamylase, α-glucosidase, β-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, and/or xylanase, or any combination thereof for removing biofilms. The additional enzyme(s) may be producible by means of a microorganism belonging to the genus Aspergillus, e.g., A. aculeatus, A. awamori, A. niger, or A. oryzae; or Trichoderma; Humicola, e.g., H. insolens; or Fusarium, e.g., F. bactridioides, F. cerealis, F. crookwellense, F. culmorum, F. graminearum, F. graminum, F. heterosporum, F. negundi, F. oxysporum, F. reticulatum, F. roseum, F. sambucinum, F. sarcochroum, F. sulphureum, F. toruloseum, F. trichothecioides, or F. venenatum.

The α-amylase variant comprising compositions may be prepared in accordance with methods known in the art and may be in the form of a liquid or a dry composition. For instance, the α-amylase variant containing composition may be in the form of a granulate or a microgranulate. The polypeptide to be included in the composition may be stabilized in accordance with methods known in the art.

Examples are given below of uses of the polypeptide compositions. The dosage of the α-amylase variant containing composition and other conditions under which the composition is used may be determined on the basis of methods known in the art. The α-amylase variants are further contemplated for use in a composition along with a 2,6-β-D-fructan hydrolase or variant thereof.

One aspect is disintegration and/or removal of biofilm. The term “disintegration” as used herein is to be understood as hydrolysis of polysaccharides in a biofilm matrix connecting and binding together individual microbial cells in the biofilm, whereby the microbial cells can be released and removed from the biofilm. The biofilm may be present at a surface, and the disintegration of the biofilm can be achieved by bringing the surface in contact with an aqueous medium, e.g., by immersing, covering or splashing, where the aqueous medium comprises an α-amylase variant and optionally one or more other enzymes responsible for breaking down biofilms, such as but not limited to 2,6-β-D-fructan hydrolase. The composition can be used to hydrolyse slime, e.g., in white waters in the pulping and paper industry.

The α-amylase variant may be present in the amount of 0.0001 to 10000 mg/L, 0.001-1000 mg/L, 0.01-100 mg/L, or even 0.1-10 mg/L. Additional enzymes and enzyme variants may be present in similar amounts or less. The process may be performed at temperatures from about ambient temperature to about 70° C. A suitable temperature range is from about 30° C. to about 60° C., e.g., about 40° C. to about 50° C.

A suitable pH for the hydrolyzing biofilms lies within from about 3.5 to about 8.5. A particularly suitable pH range is from about 5.5 to about 8, e.g. from about 6.5 to about 7.5. The contact time or reaction time for the enzyme variant to effectively removing a biofilm may vary considerably, depending on the biofilm properties and the frequency of which a surface is treated with the enzyme variant alone or in combination with other enzymes, such as 2,6-β-D-fructan hydrolase, but a suitable reaction time lies within about 0.25 to about 25 hours. A particularly suitable reaction time is from about 1 to about 10 hours, e.g., about 2 hours.

Additional enzymes can be combined with the α-amylase variants and 2,6-β-D-fructan hydrolases, including, but not limited to, cellulases, hemicellulases, xylanases, other amylases including other α-amylases, lipases, proteases, and/or pectinases. The enzymes can further be combined with antimicrobial agents such as enzymatic or non-enzymatic biocides. An enzymatic biocide may be a composition comprising an oxidoreductase, e.g., a laccase or a peroxidase, especially haloperoxidase, and optionally an enhancing agent, such as an alkyl syringate, as described in WO 97/42825 and DK 97/1273, for example.

The surface from which a biofilm is to be removed and/or cleaned off may be a hard surface, which by definition relates to any surface which is essentially non-permeable to microorganisms. Examples are surfaces made from metal, e.g., stainless steel alloys, plastics/synthetic polymers, rubber, board, glass, wood, paper, textile, concrete, rock, marble, gypsum and ceramic materials which optionally may be coated with paint, enamel, polymers and the like. Accordingly, the surface may be a member of a system holding, transporting, processing, or contacting aqueous solutions, such as water supply systems, food processing systems, cooling systems, chemical processing systems, pharmaceutical processing systems, or wood processing system, such as found in the pulp and/or paper industry. Accordingly, the enzyme variants and compositions containing the enzyme variants are useful in a conventional cleaning-in-place (C-I-P) system. The surface may a member of a system unit such as pipes, tanks, pumps, membranes, filters, heat exchangers, centrifuges, evaporators, mixers, spray towers, valves and reactors. The surface may also be or be a part of utensils used in the medical science and industry such as contaminated endoscopes, prosthetic devices or medical implants.

The compositions for biofilm removal are also contemplated for preventing so-called bio-corrosion occurring when a metal surface, e.g., a pipeline, is attacked by a microbial biofilm. The compositions disintegrate the biofilm, thereby preventing the microbial cells of the biofilm from creating a biofilm environment that would corrode the metal surface to which it is attached.

### 7.1. Oral Care Compositions

Additional applications for anti-biofilm compositions include oral care. Surfaces thus include teeth with dental plaque. Accordingly, the variant enzymes can be used for compositions, e.g., toothpaste, and processes for making a medicament comprising an enzyme variant for disintegration of plaque present on a human or animal tooth. A further use is disintegration of biofilm from mucous membranes, such as biofilm in lungs in patients suffering from cystic fibrosis. The surface also may be other surfaces of biological origin, e.g., skin, teeth, hair, nails, or may be contaminated contact lenses.

Other enzymes useful in oral care compositions include, but are not limited to, 2,6-β-D-fructan hydrolase; dextranase; mutanases; oxidases, such as glucose oxidase; L-amino acid oxidase; peroxidases, such as Coprinus sp. peroxidases described in WO 95/10602 or lactoperoxidase; haloperoxidases, especially haloperoxidase from Curvularia sp., in particular C. verruculosa and C. inaequalis; laccases; proteases, such as papain; acidic protease (e.g., the acidic proteases described in WO 95/02044); endoglucosidases; lipases; amylases, including amyloglucosidases, such as AMG™ (from Novozymes, formerly Novo Nordisk A/S); anti-microbial enzymes; and mixtures thereof. The oral care product optionally may comprise a starch binding domain such as that disclosed in U.S. Pat. No. 6,207,149.

The oral care composition may have any suitable physical form, i.e., powder, paste, gel, liquid, ointment, tablet, etc. An “oral care composition” includes a composition that can be used for maintaining or improving the oral hygiene in the mouth of humans and animals by preventing dental caries, preventing the formation of dental plaque and tartar, removing dental plaque and tartar, preventing and/or treating dental diseases, etc. Oral care compositions also encompass products for cleaning dentures, artificial teeth, and the like. Examples of oral care compositions include toothpaste, dental cream, gel or tooth powder, odontic mouth washes, pre- or post brushing rinse formulations, chewing gum, lozenges, and candy. Toothpastes and tooth gels typically include abrasive polishing materials, foaming agents, flavoring agents, humectants, binders, thickeners, sweetening agents, whitening/bleaching/stain removing agents, water, and optionally enzymes. Mouthwashes, including plaque-removing liquids, typically comprise a water/alcohol solution, flavor, humectant, sweetener, foaming agent, colorant, and optionally enzymes.

Abrasive polishing material may also be incorporated into the oral care composition. Accordingly, abrasive polishing material can include alumina and hydrates thereof, such as α-alumina trihydrate; magnesium trisilicate; magnesium carbonate; kaolin; aluminosilicates, such as calcined aluminum silicate and aluminum silicate; calcium carbonate; zirconium silicate; and also powdered plastics, such as polyvinyl chloride; polyamides; polymethyl methacrylate; polystyrene; phenol-formaldehyde resins; melamine-formaldehyde resins; urea-formaldehyde resins; epoxy resins; powdered polyethylene; silica xerogels; hydrogels and aerogels and the like. Also suitable as abrasive agents are calcium pyrophosphate; water-insoluble alkali metaphosphates; dicalcium phosphate and/or its dihydrate, dicalcium orthophosphate; tricalcium phosphate; particulate hydroxyapatite and the like. It is also possible to employ mixtures of these substances. Depending on the oral care composition, the abrasive product may be present at about 0% to about 70% by weight, for example, from about 1% to about 70%. For toothpastes, the abrasive material content typically lies in the range of 10% to 70% by weight of the final toothpaste.

Humectants are employed to prevent loss of water from tooth pastes, for example. Suitable humectants for use in oral care compositions include glycerol; polyol; sorbitol; polyethylene glycols (PEG); propylene glycol; 1,3-propanediol; 1,4-butanediol; hydrogenated partially hydrolyzed polysaccharides and the like and mixtures thereof. Humectants are in general present at 0% to about 80% or about 5% to about 70% by weight in toothpaste.

Silica, starch, tragacanth gum, xanthan gum, extracts of Irish moss, alginates, pectin, cellulose derivatives, such as hydroxyethyl cellulose, sodium carboxymethyl cellulose and hydroxypropyl cellulose, polyacrylic acid and its salts, polyvinylpyrrolidone, are examples of suitable thickeners and binders that help stabilize a dentifrice product. Thickeners may be present in toothpaste creams and gels at about 0.1% to about 20% by weight, and binders at about 0.01 to about 10% by weight of the final product.

A foaming agent can be used, including soap, anionic, cationic, non-ionic, amphoteric and/or zwitterionic surfactants. These may be present at levels of 0% to about 15%, about 0.1 to about 13%, or even about 0.25% to about 10% by weight of the final product. Surfactants are only suitable to the extent that they do not inactivate the present enzymes. Surfactants include fatty alcohol sulfates, salts of sulphonated mono-glycerides or fatty acids having 10 to 20 carbon atoms, fatty acid-albumen condensation products, salts of fatty acids amides and taurines, and/or salts of fatty acid esters of isethionic acid.

Suitable sweeteners include saccharin for use in a formulation. Flavors, such as spearmint, also are usually present in low amounts, such as from about 0.01% to about 5% by weight, especially from about 0.1% to about 5%. Whitening/bleaching agents include H₂O₂ and may be added in amounts less than about 5% or from about 0.25% to about 4%, calculated by the weight of the final product. The whitening/bleaching agents may be an enzyme, such as an oxidoreductase. Examples of suitable teeth bleaching enzymes are described in WO 97/06775 (Novo Nordisk A/S). Water is usually added in an amount giving the composition, e.g. toothpaste, a flowable form. Water-soluble anti-bacterial agents, such as chlorhexidine digluconate, hexetidine, alexidine, Triclosan®, quaternary ammonium anti-bacterial compounds and water-soluble sources of certain metal ions such as zinc, copper, silver and stannous (e.g., zinc, copper and stannous chloride, and silver nitrate) also may be included. Additional compounds that can be used include a fluoride source, dyes/colorants, preservatives, vitamins, pH-adjusting agents, anti-caries agents, desensitizing agents, etc.

Enzymes are also useful in the oral care compositions described above. Enzymes provide several benefits when used for cleansing of the oral cavity. Proteases break down salivary proteins, which are adsorbed onto the tooth surface and form the pellicle, the first layer of resulting plaque. Proteases along with lipases destroy bacteria by lysing proteins and lipids which form the structural components of bacterial cell walls and membranes. Dextranase and other carbohydrases, such as the 2,6-β-D-fructan hydrolase, break down the organic skeletal structure produced by bacteria that forms a matrix for bacterial adhesion. Proteases and amylases not only prevent plaque formation, but also prevent the development of mineralization by breaking-up carbohydrate-protein complexes that bind calcium.

A toothpaste may typically comprise the following ingredients (in weight % of the final toothpaste composition): abrasive material to about 70%; humectant: 0% to about 80%; thickener: about 0.1% to about 20%; binder: about 0.01% to about 10%; sweetener: about 0.1% to about 5%; foaming agent: 0% to about 15%; whitener: 0% to about 5%; and enzymes: about 0.0001% to about 20%. In one embodiment, a toothpaste has a pH in the range from about 6.0 to about 8.0, and comprises: about 10% to about 70% abrasive material; 0% to about 80% humectant; 0.1% to about 20% thickener; 0.01% to about 10% binder; about 0.1% to about 5% sweetener; 0% to about 15% foaming agent; 0% to about 5% whitener; and about 0.0001% to about 20% enzymes. These enzymes include α-amylase variants alone or in combination with other enzymes, such as 2,6-β-D-fructan hydrolase, and optionally other types of enzymes mentioned above.

A mouthwash typically may comprise the following ingredients (in weight % of the final mouth wash composition): 0% to about 20% humectant; 0% to about 2% surfactant; 0% to about 5% enzymes; 0% to about 20% ethanol; 0% to about 2% other ingredients (e.g., flavor, sweetener active ingredients such as fluorides). The composition can also contain from about 0% to about 70% water. The mouthwash composition may be buffered with an appropriate buffer, e.g. sodium citrate or phosphate in the pH-range of about 6.0 to about 7.5. The mouthwash may be in none-diluted form, i.e., it should be diluted before use. The oral care compositions may be produced using any conventional method known to the art of oral care.

### 8. STARCH PROCESSING COMPOSITIONS AND USE

In another aspect, compositions with the disclosed α-amylase variants can be utilized for starch liquefaction and/or saccharification. Starch processing is useful for producing sweetener; producing alcohol for fuel or drinking (i.e., potable alcohol), producing a beverage, processing cane sugar, or producing desired organic compounds, e.g., citric acid, itaconic acid, lactic acid, gluconic acid, ketones, amino acids, antibiotics, enzymes, vitamins, and hormones. Conversion of starch to fructose syrups normally consists of three consecutive enzymatic processes: a liquefaction process, a saccharification process, and an isomerization process. During the liquefaction process, a variant B. licheniformis α-amylase degrades starch to dextrins at a pH between about pH 5.5 and about pH 6.2 and at temperatures of about 95° C. to about 160° C. for a period of approximately 2 hours. About 1 mM of calcium (40 ppm free calcium ions) typically is added to optimize enzyme stability under these conditions. Other α-amylase variants may require different conditions.

After the liquefaction process, the dextrins can be converted into dextrose by addition of a glucoamylase (e.g., AMG™) and optionally a debranching enzyme, such as an isoamylase or a pullulanase (e.g., Promozyme®). Before this step, the pH is reduced to a value below about 4.5, maintaining the high temperature (above 95° C.), and the liquefying α-amylase variant activity is denatured. The temperature is lowered to 60° C., and a glucoamylase and a debranching enzyme can be added. The saccharification process proceeds typically for about 24 to about 72 hours.

After the saccharification process, the pH is increased to a value in the range of about 6.0 to about 8.0, e.g., pH 7.5, and the calcium is removed by ion exchange. The dextrose syrup is then converted into high fructose syrup using an immobilized glucose isomerase (such as Sweetzyme®), for example.

The α-amylase variant may provide at least one improved enzymatic property for conducting the process of liquefaction. For example, the variant α-amylase may have a higher activity, or it may have a reduced requirement for calcium. Addition of free calcium is required to ensure adequately high stability of the α-amylase; however, free calcium strongly inhibits the activity of the glucose isomerase. Accordingly, the calcium should be removed prior to the isomerization step, by means of an expensive unit operation, to an extent that reduces the level of free calcium to below 3-5 ppm. Cost savings can be obtained if such an operation could be avoided, and the liquefaction process could be performed without addition of free calcium ions. Thus, α-amylase variants that do not require calcium ions or that have a reduced requirement for calcium are particularly advantageous. For example, a less calcium-dependent α-amylase variant, which is stable and highly active at low concentrations of free calcium (<40 ppm) can be utilized in the composition and procedures. Such an α-amylase variant should have a pH optimum in the range of about 4.5 to about 6.5, e.g., about pH 4.5 to about pH 5.5. The α-amylase variants can be used alone to provide specific hydrolysis or can be combined with other amylases to provide a “cocktail” with a broad spectrum of activity.

The starch to be processed may be a highly refined starch quality, for instance, at least 90%, at least 95%, at least 97%, or at least 99.5% pure. Alternatively, the starch can be a more crude starch containing material comprising milled whole grain, including non-starch fractions such as germ residues and fibers. The raw material, such as whole grain, is milled to open up the structure and allow further processing. Two milling processes are suitable: wet and dry milling. Also, corn grits, and milled corn grits may be applied. Dry milled grain will comprise significant amounts of non-starch carbohydrate compounds, in addition to starch. When such a heterogeneous material is processed by jet cooking, often only a partial gelatinization of the starch is achieved. Accordingly, α-amylase variants having a high activity towards ungelatinized starch are advantageously applied in a process comprising liquefaction and/or saccharification jet cooked dry milled starch.

A variant α-amylase having a superior hydrolysis activity during the liquefaction process advantageously increases the efficiency of the saccharification step (see WO 98/22613 (Novo Nordisk A/S)) and the need for glucoamylase during the saccharification step. The glucoamylase advantageously is present in an amount of no more than, or even less than, 0.5 glucoamylase activity unit (AGU)/g DS (i.e., glucoamylase activity units per gram of dry solids). The glucoamylase may be derived from a strain within Aspergillus sp., Talaromyces sp., Pachykytospora sp., or Trametes sp., with exemplary examples being Aspergillus niger, Talaromyces emersonii, Trametes cingulata, or Pachykytospora papyracea. In one embodiment, the process also comprises the use of a carbohydrate-binding domain of the type disclosed in WO 98/22613.

In yet another aspect, the process may comprise hydrolysis of a slurry of gelatinized or granular starch, in particular hydrolysis of granular starch into a soluble starch hydrolysate at a temperature below the initial gelatinization temperature of the granular starch. In addition to being contacted with an α-amylase variant, the starch may be contacted with one or more enzyme selected from the group consisting of a fungal α-amylase (EC 3.2.1.1), a β-amylase (EC 3.2.1.2), and a glucoamylase (EC 3.2.1.3). In an embodiment further another amylolytic enzyme or a debranching enzyme, such as an isoamylase (EC 3.2.1.68), or a pullulanases (EC 3.2.1.41) may be added to the α-amylase variant.

In one embodiment, the process is conducted at a temperature below the initial gelatinization temperature. Such processes are often conducted at least at 30° C., at least 31° C., at least 32° C., at least 33° C., at least 34° C., at least 35° C., at least 36° C., at least 37° C., at least 38° C., at least 39° C., at least 40° C., at least 41° C., at least 42° C., at least 43° C., at least 44° C., at least 45° C., at least 46° C., at least 47° C., at least 48° C., at least 49° C., at least 50° C., at least 51° C., at least 52° C., at least 53° C., at least 54° C., at least 55° C., at least 56° C., at least 57° C., at least 58° C., at least 59° C., or at least 60° C. The pH at which the process is conducted may in be in the range of about 3.0 to about 7.0, from about 3.5 to about 6.0, or from about 4.0 to about 5.0. One aspect contemplates a process comprising fermentation with a yeast, for example, to produce ethanol at a temperature around 32° C., such as from 30° C. to 35° C. In another aspect, the process comprises simultaneous saccharification and fermentation with a yeast to produce ethanol or with another suitable fermentation organism to produce a desired organic compound, for example, at a temperature from 30° C. to 35° C., e.g., at around 32° C. In the above fermentation processes, the ethanol content reaches at least about 7%, at least about 8%, at least about 9%, at least about 10%, at least about 11%, at least about 12%, at least about 13%, at least about 14%, at least about 15%, or at least about 16% ethanol.

The starch slurry to be used in any of the above aspects may have about 20% to about 55% dry solids granular starch, about 25% to about 40% dry solids granular starch, or about 30% to about 35% dry solids granular starch. The enzyme variant converts the soluble starch into a soluble starch hydrolysate of the granular starch in the amount of at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

In another embodiment, the α-amylase variant is used in a process for liquefaction or saccharification of a gelatinized starch, including, but not limited to, gelatinization by jet cooking. The process may comprise fermentation to produce a fermentation product, e.g., ethanol. Such a process for producing ethanol from starch-containing material by fermentation comprises: (i) liquefying the starch-containing material with an α-amylase variant; (ii) saccharifying the liquefied mash obtained; and (iii) fermenting the material obtained in step (ii) in the presence of a fermenting organism. Optionally the process further comprises recovery of the ethanol. The saccharification and fermentation processes may be carried out as a simultaneous saccharification and fermentation (SSF) process. During the fermentation, the ethanol content reaches at least about 7%, at least about 8%, at least about 9%, at least about 10% such as at least about 11%, at least about 12%, at least about 13%, at least about 14%, at least 15%, or at least 16% ethanol.

The starch to be processed in the above aspects may be obtained from tubers, roots, stems, legumes, cereals or whole grain. More specifically, the granular starch may be obtained from corns, cobs, wheat, barley, rye, milo, sago, cassaya, tapioca, sorghum, rice, peas, bean, banana, or potatoes. Specially contemplated are both waxy and non-waxy types of corn and barley.

As used herein, the term “liquefaction” or “liquefy” means a process by which starch is converted to less viscous and shorter chain dextrins. Generally, this process involves gelatinization of starch simultaneously with or followed by the addition of an α-amylase variant. Additional liquefaction-inducing enzymes optionally may be added. As used herein, the term “primary liquefaction” refers to a step of liquefaction when the slurry's temperature is raised to or near its gelatinization temperature. Subsequent to the raising of the temperature, the slurry is sent through a heat exchanger or jet to temperatures from about 90-150° C., e.g., 100-110° C. Subsequent to application to a heat exchange or jet temperature, the slurry is held for a period of 3-10 minutes at that temperature. This step of holding the slurry at 90-150° C. is termed primary liquefaction.

As used herein, the term “secondary liquefaction” refers the liquefaction step subsequent to primary liquefaction (heating to 90-150° C.), when the slurry is allowed to cool to room temperature. This cooling step can be 30 minutes to 180 minutes, e.g. 90 minutes to 120 minutes. As used herein, the term “minutes of secondary liquefaction” refers to the time that has elapsed from the start of secondary liquefaction to the time that the Dextrose Equivalent (DE) is measured.

Another aspect contemplates the additional use of a β-amylase in the composition comprising the α-amylase variant. β-amylases (EC 3.2.1.2) are exo-acting maltogenic amylases, which catalyze the hydrolysis of 1,4-α-glucosidic linkages into amylose, amylopectin, and related glucose polymers, thereby releasing maltose. β-amylases have been isolated from various plants and microorganisms (Fogarty et al., PROGRESS IN INDUSTRIAL MICROBIOLOGY, Vol. 15, pp. 112-115, 1979). These β-amylases are characterized by having optimum temperatures in the range from 40° C. to 65° C., and optimum pH in the range from about 4.5 to about 7.0. Contemplated β-amylases include, but are not limited to, β-amylases from barley Spezyme® BBA 1500, Spezyme® DBA, Optimalt™ ME, Optimalt™ BBA (Danisco US Inc., Genencor Division; previously known as Genencor International, Inc.); and Novozym™ WBA (Novozymes A/S).

Another enzyme contemplated for use in the composition is a glucoamylase (EC 3.2.1.3). Glucoamylases are derived from a microorganism or a plant. For example, glucoamylases can be of fungal or bacterial origin. Exemplary bacterial glucoamylases are Aspergillus glucoamylases, in particular A. niger G1 or G2 glucoamylase (Boel et al. (1984), EMBO J. 3(5): 1097-1102), or variants thereof, such as disclosed in WO 92/00381 and WO 00/04136; A. awamori glucoamylase (WO 84/02921); A. oryzae glucoamylase (Agric. Biol. Chem. (1991), 55(4): 941-949), or variants or fragments thereof.

Other contemplated Aspergillus glucoamylase variants include variants to enhance the thermal stability: G137A and G139A (Chen et al. (1996), Prot. Eng. 9: 499-505); D257E and D293E/Q (Chen et al. (1995), Prot. Eng. 8: 575-582); N182 (Chen et al. (1994), Biochem. J. 301: 275-281); disulphide bonds, A246C (Fierobe et al. (1996), Biochemistry, 35: 8698-8704); and introduction of Pro residues in positions A435 and 5436 (Li et al. (1997) Protein Eng. 10: 1199-1204). Other contemplated glucoamylases include Talaromyces glucoamylases, in particular derived from T. emersonii (WO 99/28448), T. leycettanus (U.S. Pat. No. RE 32, 153), T. duponti, or T. thermophilus (U.S. Pat. No. 4,587,215). Contemplated bacterial glucoamylases include glucoamylases from the genus Clostridium, in particular C. thermoamylolyticum (EP 135138) and C. thermohydrosulfuricum (WO 86/01831). Suitable glucoamylases include the glucoamylases derived from Aspergillus oryzae, such as a glucoamylase having 50%, 55%, 60%, 65%, 70%, 75%, 80%, 85%, or even 90% homology to the amino acid sequence shown in SEQ ID NO:2 in WO 00/04136. Also suitable are commercial glucoamylases, such as AMG 200L; AMG 300 L; SAN™ SUPER and AMG™ E (from Novozymes); OPTIDEX® 300 (from Danisco US Inc., Genencor Division; previously known as Genencor International, Inc.); AMIGASE™ and AMIGASE™ PLUS (from DSM); G-ZYME® G900 (from Enzyme Bio-Systems); and G-ZYME® G990 ZR (A. niger glucoamylase and low protease content). Glucoamylases may be added in an amount of 0.02-2.0 AGU/g DS or 0.1-1.0 AGU/g DS, e.g., 0.2 AGU/g DS.

Additional enzyme variants can be included in the composition. Two or more α-amylase variants can be used alone or in combination with other enzymes discussed herein. For example, a third enzyme may be another α-amylase, e.g., a yeast α-amylase, or another α-amylase variant. These can be Bacillus α-amylases or non-Bacillus α-amylases.

Another enzyme that can optionally be added is a debranching enzyme, such as an isoamylase (EC 3.2.1.68) or a pullulanases (EC 3.2.1.41). Isoamylase hydrolyses α-1,6-D-glucosidic branch linkages in amylopectin and β-limit dextrins and can be distinguished from pullulanases by the inability of isoamylase to attack pullulan and by the limited action of isoamylase on α-limit dextrins. Debranching enzymes may be added in effective amounts well known to the person skilled in the art.

The exact composition of the products of the process depends on the combination of enzymes applied, as well as the type of granular starch processed. The soluble hydrolysate may be maltose with a purity of at least about 85%, at least about 90%, at least about 95.0%, at least about 95.5%, at least about 96.0%, at least about 96.5%, at least about 97.0%, at least about 97.5%, at least about 98.0%, at least about 98.5%, at least about 99.0% or at least about 99.5%. Alternatively, the soluble starch hydrolysate is glucose, or the starch hydrolysate has a DE (glucose percent of total solubilized dry solids) of at least 94.5%, at least 95.0%, at least 95.5%, at least 96.0%, at least 96.5%, at least 97.0%, at least 97.5%, at least 98.0%, at least 98.5%, at least 99.0% or at least 99.5%. In one embodiment, a process of manufacturing ice creams, cakes, candies, canned fruit uses a specialty syrup containing a mixture of glucose, maltose, DP3 and DPn.

Two milling processes are suitable: wet milling and dry milling. In dry milling, the whole kernel is milled and used. Wet milling gives a good separation of germ and meal (starch granules and protein) and is usually used when the starch hydrolysate is used in production of syrups. Both dry and wet milling are well known in the art of starch processing and also are contemplated for use with the compositions and methods disclosed. The process may be conducted in an ultrafiltration system where the retentate is held under recirculation in presence of enzymes, raw starch and water, where the permeate is the soluble starch hydrolysate. Another method is the process conducted in a continuous membrane reactor with ultrafiltration membranes, where the retentate is held under recirculation in presence of enzymes, raw starch and water, and where the permeate is the soluble starch hydrolysate. Also contemplated is the process conducted in a continuous membrane reactor with microfiltration membranes and where the retentate is held under recirculation in presence of enzymes, raw starch and water, and where the permeate is the soluble starch hydrolysate.

In one regard, the soluble starch hydrolysate of the process is subjected to conversion into high fructose starch-based syrup (HFSS), such as high fructose corn syrup (HFCS). This conversion can be achieved using a glucose isomerase, particularly a glucose isomerase immobilized on a solid support. Contemplated isomerases included the commercial products Sweetzyme®, IT (Novozymes A/S); G-zyme® IMGI, and G-zyme® G993, Ketomax®, G-zyme® G993, G-zyme® G993 liquid, and GenSweet® IGI.

In another aspect, the soluble starch hydrolysate of produced yields production of fuel or potable ethanol. In the process of the third aspect the fermentation may be carried out simultaneously or separately/sequential to the hydrolysis of the granular starch slurry. When the fermentation is performed simultaneously with the hydrolysis, the temperature can be between 30° C. and 35° C., particularly between 31° C. and 34° C. The process may be conducted in an ultrafiltration system where the retentate is held under recirculation in presence of enzymes, raw starch, yeast, yeast nutrients and water and where the permeate is an ethanol containing liquid. Also contemplated is the process conducted in a continuous membrane reactor with ultrafiltration membranes and where the retentate is held under recirculation in presence of enzymes, raw starch, yeast, yeast nutrients and water and where the permeate is an ethanol containing liquid.

The soluble starch hydrolysate of the process may also be used for production of a fermentation product comprising fermenting the treated starch into a fermentation product, such as citric acid, monosodium glutamate, gluconic acid, sodium gluconate, calcium gluconate, potassium gluconate, glucono delta-lactone, or sodium erythorbate.

The amylolytic activity of the α-amylase variant may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

### 9. TEXTILE DESIZING COMPOSITIONS AND USE

Also contemplated are compositions and methods of treating fabrics (e.g., to desize a textile) using one or more of the α-amylase variants. The α-amylase variants can be used in any fabric-treating method, which are well known in the art (see, e.g., U.S. Pat. No. 6,077,316). For example, in one aspect, the feel and appearance of a fabric is improved by a method comprising contacting the fabric with an enzyme variant in a solution. In one aspect, the fabric is treated with the solution under pressure.

In one aspect, the enzymes are applied during or after the weaving of textiles, or during the desizing stage, or one or more additional fabric processing steps. During the weaving of textiles, the threads are exposed to considerable mechanical strain. Prior to weaving on mechanical looms, warp yarns are often coated with sizing starch or starch derivatives in order to increase their tensile strength and to prevent breaking. The α-amylase variant can be applied to remove these sizing starch or starch derivatives. After the textiles have been woven, a fabric can proceed to a desizing stage. This can be followed by one or more additional fabric processing steps. Desizing is the act of removing size from textiles. After weaving, the size coating should be removed before further processing the fabric in order to ensure a homogeneous and wash-proof result. Also provided is a method of desizing comprising enzymatic hydrolysis of the size by the action of an enzyme variant.

The α-amylase variant can be used alone or with other desizing chemical reagents and/or desizing enzymes to desize fabrics, including cotton-containing fabrics, as detergent additives, e.g., in aqueous compositions. The α-amylase variant can also be used in compositions and methods for producing a stonewashed look on indigo-dyed denim fabric and garments. For the manufacture of clothes, the fabric can be cut and sewn into clothes or garments, which are afterwards finished. In particular, for the manufacture of denim jeans, different enzymatic finishing methods have been developed. The finishing of denim garment normally is initiated with an enzymatic desizing step, during which garments are subjected to the action of amylolytic enzymes to provide softness to the fabric and make the cotton more accessible to the subsequent enzymatic finishing steps. The α-amylase variant can be used in methods of finishing denim garments (e.g., a “bio-stoning process”), enzymatic desizing and providing softness to fabrics, and/or finishing process.

### 10. COMPOSITIONS AND METHODS FOR BAKING AND FOOD PREPARATION

For the commercial and home use of flour for baking and food production, it is important to maintain an appropriate level of α-amylase activity in the flour. A level of activity that is too high may result in a product that is sticky and/or doughy and unmarketable; but flour with insufficient α-amylase activity may not contain enough sugar for proper yeast function, resulting in dry, crumbly bread. Accordingly, a B. licheniformis α-amylase variant, by itself or in combination with another α-amylase(s), may be added to the flour to augment the level of endogenous α-amylase activity in flour. The α-amylase typically has a temperature optimum in the presence of starch in the ranges of 30-90° C., 50-80° C., 55-75° C., or 60-70° C., for example. The temperature optimum may be measured in a 1% solution of soluble starch at pH 5.5.

In addition to the use of grains and other plant products in baking, grains such as barley, oats, wheat, as well as plant components, such as corn, hops, and rice are used for brewing, both in industry and for home brewing. The components used in brewing may be unmalted or may be malted, i.e., partially germinated, resulting in an increase in the levels of enzymes, including α-amylase. For successful brewing, adequate levels of α-amylase enzyme activity are necessary to ensure the appropriate levels of sugars for fermentation. A B. licheniformis α-amylase variant, by itself or in combination with another α-amylase(s), accordingly may be added to the components used for brewing.

As used herein, the term “flour” means milled or ground cereal grain. The term “flour” also may mean Sago or tuber products that have been ground or mashed. In some embodiments, flour may also contain components in addition to the milled or mashed cereal or plant matter. An example of an additional component, although not intended to be limiting, is a leavening agent. Cereal grains include wheat, oat, rye, and barley. Tuber products include tapioca flour, cassaya flour, and custard powder. The term “flour” also includes ground corn flour, maize-meal, rice flour, whole-meal flour, self-rising flour, tapioca flour, cassaya flour, ground rice, enriched flower, and custard powder.

As used herein, the term “stock” means grains and plant components that are crushed or broken. For example, barley used in beer production is a grain that has been coarsely ground or crushed to yield a consistency appropriate for producing a mash for fermentation. As used herein, the term “stock” includes any of the aforementioned types of plants and grains in crushed or coarsely ground forms. The methods described herein may be used to determine α-amylase activity levels in both flours and stock.

A B. licheniformis α-amylase variant further can be added alone or in a combination with other amylases to prevent or retard staling, i.e., crumb firming of baked products. The amount of anti-staling amylase will typically be in the range of 0.01-10 mg of enzyme protein per kg of flour, e.g., 1-10 mg/kg. Additional anti-staling amylases that can be used in combination with a B. licheniformis α-amylase variant include an endo-amylase, e.g., a bacterial endo-amylase from Bacillus. The additional amylase can be a maltogenic α-amylase (EC 3.2.1.133), e.g., from Bacillus. Novamyl® is a suitable maltogenic α-amylase from B. stearothermophilus strain NCIB 11837 and is described in Christophersen et al., Starch, 50(1): 39-45 (1997). Other examples of anti-staling endo-amylases include bacterial α-amylases derived from Bacillus, such as B. licheniformis or B. amyloliquefaciens. The anti-staling amylase may be an exo-amylase, such as β-amylase, e.g., from plant sources, such as soy bean, or from microbial sources, such as Bacillus.

The baking composition comprising a B. licheniformis α-amylase variant further can comprise a phospholipase. The phospholipase may have A₁ or A₂ activity to remove fatty acid from the phospholipids, forming a lyso-phospholipid. It may or may not have lipase activity, i.e., activity on triglycerides. The phospholipase typically has a temperature optimum in the range of 30-90° C., e.g., 30-70° C. The added phospholipases can be of animal origin, for example, from pancreas, e.g., bovine or porcine pancreas, snake venom or bee venom. Alternatively, the phospholipase may be of microbial origin, e.g., from filamentous fungi, yeast or bacteria, such as the genus or species Aspergillus, A. niger; Dictyostelium, D. discoideum; Mucor, M. javanicus, M. mucedo, M. subtilissimus; Neurospora, N. crassa; Rhizomucor, R. pusillus; Rhizopus, R. arrhizus, R. japonicus, R. stolonifer; Sclerotinia, S. libertiana; Trichophyton, T. rubrum; Whetzelinia, W. sclerotiorum; Bacillus, B. megaterium, B. subtilis; Citrobacter, C. freundii; Enterobacter, E. aerogenes, E. cloacae; Edwardsiella, E. tarda; Etwinia, E. herbicola; Escherichia, E. coli; Klebsiella, K. pneumoniae; Proteus, P. vulgaris; Providencia, P. stuartii; Salmonella, S. typhimurium; Serratia, S. liquefasciens, S. marcescens; Shigella, S. flexneri; Streptomyces, S. violeceoruber; Yersinia, Y. enterocolitica; Fusarium, F. oxysporum, strain DSM 2672), for example.

A phospholipase is added in an amount that improves the softness of the bread during the initial period after baking, particularly the first 24 hours. The amount of phospholipase will typically be in the range of 0.01-10 mg of enzyme protein per kg of flour, e.g., 0.1-5 mg/kg. That is, phospholipase activity generally will be in the range of 20-1000 Lipase Unit (LU)/kg of flour, where a Lipase Unit is defined as the amount of enzyme required to release 1 μmol butyric acid per minute at 30° C., pH 7.0, with gum arabic as emulsifier and tributyrin as substrate.

Compositions of dough generally comprise wheat meal or wheat flour and/or other types of meal, flour or starch such as corn flour, cornstarch, rye meal, rye flour, oat flour, oatmeal, soy flour, sorghum meal, sorghum flour, potato meal, potato flour or potato starch. The dough may be fresh, frozen or par-baked. The dough can be a leavened dough or a dough to be subjected to leavening. The dough may be leavened in various ways, such as by adding chemical leavening agents, e.g., sodium bicarbonate or by adding a leaven, i.e., fermenting dough. Dough also may be leavened by adding a suitable yeast culture, such as a culture of Saccharomyces cerevisiae (baker's yeast), e.g., a commercially available strain of S. cerevisiae.

The dough may also comprise other conventional dough ingredients, e.g., proteins, such as milk powder, gluten, and soy; eggs (e.g., whole eggs, egg yolks or egg whites); an oxidant, such as ascorbic acid, potassium bromate, potassium iodate, azodicarbonamide (ADA) or ammonium persulfate; an amino acid such as L-cysteine; a sugar; or a salt, such as sodium chloride, calcium acetate, sodium sulfate or calcium sulfate. The dough further may comprise fat, e.g., triglyceride, such as granulated fat or shortening. The dough further may comprise an emulsifier such as mono- or diglycerides, diacetyl tartaric acid esters of mono- or diglycerides, sugar esters of fatty acids, polyglycerol esters of fatty acids, lactic acid esters of monoglycerides, acetic acid esters of monoglycerides, polyoxyethylene stearates, or lysolecithin. In particular, the dough can be made without addition of emulsifiers.

Optionally, an additional enzyme may be used together with the anti-staling amylase and the phospholipase. The additional enzyme may be a second amylase, such as an amyloglucosidase, a β-amylase, a cyclodextrin glucanotransferase, or the additional enzyme may be a peptidase, in particular an exopeptidase, a transglutaminase, a lipase, a cellulase, a hemicellulase, in particular a pentosanase such as xylanase, a protease, a protein disulfide isomerase, e.g., a protein disulfide isomerase as disclosed in WO 95/00636, for example, a glycosyltransferase, a branching enzyme (1,4-α-glucan branching enzyme), a 4-α-glucanotransferase (dextrin glycosyltransferase) or an oxidoreductase, e.g., a peroxidase, a laccase, a glucose oxidase, a pyranose oxidase, a lipoxygenase, an L-amino acid oxidase or a carbohydrate oxidase. The additional enzyme(s) may be of any origin, including mammalian and plant, and particularly of microbial (bacterial, yeast or fungal) origin and may be obtained by techniques conventionally used in the art.

The xylanase is typically of microbial origin, e.g., derived from a bacterium or fungus, such as a strain of Aspergillus, in particular of A. aculeatus, A. niger (cf. WO 91/19782), A. awamori (e.g., WO 91/18977), or A. tubigensis (e.g., WO 92/01793); from a strain of Trichoderma, e.g., T. reesei, or from a strain of Humicola, e.g., H. insolens (e.g., WO 92/17573). Pentopan® and Novozym 384® are commercially available xylanase preparations produced from Trichoderma reesei. The amyloglucosidase may be an A. niger amyloglucosidase (such as AMG®). Other useful amylase products include Grindamyl® A 1000 or A 5000 (available from Grindsted Products, Denmark). The glucose oxidase may be a fungal glucose oxidase, in particular an Aspergillus niger glucose oxidase (such as Gluzyme®). An exemplary protease is. Neutrase®. An exemplary lipase can be derived from strains of Thermomyces (Humicola), Rhizomucor, Candida, Aspergillus, Rhizopus, or Pseudomonas, in particular from Thermomyces lanuginosus (Humicola lanuginosa), Rhizomucor miehei, Candida antarctica, Aspergillus niger, Rhizopus delemar or Rhizopus arrhizus or Pseudomonas cepacia. In specific embodiments, the lipase may be Lipase A or Lipase B derived from Candida antarctica as described in WO 88/02775, for example, or the lipase may be derived from Rhizomucor miehei as described in EP 238,023, for example, or Humicola lanuginosa, described in EP 305,216, for example, or Pseudomonas cepacia as described in EP 214,761 and WO 89/01032, for example.

The process may be used for any kind of baked product prepared from dough, e.g., a soft or a crisp character, or a white, light or dark type. Examples are bread, particularly white, whole-meal or rye bread, typically in the form of loaves or rolls, French baguette-type bread, pita bread, tortillas, cakes, pancakes, biscuits, cookies, pie crusts, crisp bread, steamed bread, pizza and the like.

In another embodiment, a B. licheniformis α-amylase variant may be used in a pre-mix, comprising flour together with an anti-staling amylase, a phospholipase and a phospholipid. The pre-mix may contain other dough-improving and/or bread-improving additives, e.g., any of the additives, including enzymes, mentioned above. In one aspect, the B. licheniformis α-amylase variant is a component of an enzyme preparation comprising an anti-staling amylase and a phospholipase, for use as a baking additive.

The enzyme preparation is optionally in the form of a granulate or agglomerated powder. The preparation can have a narrow particle size distribution with more than 95% (by weight) of the particles in the range from 25 to 500 μm. Granulates and agglomerated powders may be prepared by conventional methods, e.g., by spraying the B. licheniformis α-amylase variant onto a carrier in a fluid-bed granulator. The carrier may consist of particulate cores having a suitable particle size. The carrier may be soluble or insoluble, e.g., a salt (such as NaCl or sodium sulfate), a sugar (such as sucrose or lactose), a sugar alcohol (such as sorbitol), starch, rice, corn grits, or soy.

Another aspect contemplates the enveloping of particles comprising a B. licheniformis α-amylase variant, i.e., α-amylase particles. To prepare the enveloped α-amylase particles, the enzyme is contacted with a food grade lipid in sufficient quantity to suspend all of the α-amylase particles. Food grade lipids, as used herein, may be any naturally organic compound that is insoluble in water but is soluble in non-polar organic solvents such as hydrocarbon or diethyl ether. Suitable food grade lipids include, but are not limited to, triglycerides either in the form of fats or oils that are either saturated or unsaturated. Examples of fatty acids and combinations thereof which make up the saturated triglycerides include, but are not limited to, butyric (derived from milk fat), palmitic (derived from animal and plant fat), and/or stearic (derived from animal and plant fat). Examples of fatty acids and combinations thereof which make up the unsaturated triglycerides include, but are not limited to, palmitoleic (derived from animal and plant fat), oleic (derived from animal and plant fat), linoleic (derived from plant oils), and/or linolenic (derived from linseed oil). Other suitable food grade lipids include, but are not limited to, monoglycerides and diglycerides derived from the triglycerides discussed above, phospholipids and glycolipids.

The food grade lipid, particularly in the liquid form, is contacted with a powdered form of the α-amylase particles in such a fashion that the lipid material covers at least a portion of the surface of at least a majority, e.g., 100% of the α-amylase particles. Thus, each α-amylase particle is individually enveloped in a lipid. For example, all or substantially all of the α-amylase particles are provided with a thin, continuous, enveloping film of lipid. This can be accomplished by first pouring a quantity of lipid into a container, and then slurrying the α-amylase particles so that the lipid thoroughly wets the surface of each α-amylase particle. After a short period of stirring, the enveloped α-amylase particles, carrying a substantial amount of the lipids on their surfaces, are recovered. The thickness of the coating so applied to the particles of α-amylase can be controlled by selection of the type of lipid used and by repeating the operation in order to build up a thicker film, when desired.

The storing, handling and incorporation of the loaded delivery vehicle can be accomplished by means of a packaged mix. The packaged mix can comprise the enveloped α-amylase. However, the packaged mix may further contain additional ingredients as required by the manufacturer or baker. After the enveloped α-amylase has been incorporated into the dough, the baker continues through the normal production process for that product.

The advantages of enveloping the α-amylase particles are two-fold. First, the food grade lipid protects the enzyme from thermal denaturation during the baking process for those enzymes that are heat labile. Consequently, while the α-amylase is stabilized and protected during the proving and baking stages, it is released from the protective coating in the final baked good product, where it hydrolyzes the glucosidic linkages in polyglucans. The loaded delivery vehicle also provides a sustained release of the active enzyme into the baked good. That is, following the baking process, active α-amylase is continually released from the protective coating at a rate that counteracts, and therefore reduces the rate of, staling mechanisms.

In general, the amount of lipid applied to the α-amylase particles can vary from a few percent of the total weight of the α-amylase to many times that weight, depending upon the nature of the lipid, the manner in which it is applied to the α-amylase particles, the composition of the dough mixture to be treated, and the severity of the dough-mixing operation involved.

The loaded delivery vehicle, i.e., the lipid-enveloped enzyme, is added to the ingredients used to prepare a baked good in an effective amount to extend the shelf-life of the baked good. The baker computes the amount of enveloped α-amylase, prepared as discussed above, that will be required to achieve the desired anti-staling effect. The amount of the enveloped α-amylase required is calculated based on the concentration of enzyme enveloped and on the proportion of α-amylase to flour specified. A wide range of concentrations has been found to be effective, although, as has been discussed, observable improvements in anti-staling do not correspond linearly with the α-amylase concentration, but above certain minimal levels, large increases in α-amylase concentration produce little additional improvement. The α-amylase concentration actually used in a particular bakery production could be much higher than the minimum necessary in order to provide the baker with some insurance against inadvertent under-measurement errors by the baker. The lower limit of enzyme concentration is determined by the minimum anti-staling effect the baker wishes to achieve.

A method of preparing a baked good may comprise: a) preparing lipid-coated α-amylase particles, wherein substantially 100 percent of the α-amylase particles are coated; b) mixing a dough containing flour; c) adding the lipid-coated α-amylase to the dough before the mixing is complete and terminating the mixing before the lipid coating is removed from the α-amylase; d) proofing the dough; and e) baking the dough to provide the baked good, wherein the α-amylase is inactive during the mixing, proofing and baking stages and is active in the baked good.

The enveloped α-amylase can be added to the dough during the mix cycle, e.g., near the end of the mix cycle. The enveloped α-amylase is added at a point in the mixing stage that allows sufficient distribution of the enveloped α-amylase throughout the dough; however, the mixing stage is terminated before the protective coating becomes stripped from the α-amylase particle(s). Depending on the type and volume of dough, and mixer action and speed, anywhere from one to six minutes or more might be required to mix the enveloped α-amylase into the dough, but two to four minutes is average. Thus, several variables may determine the precise procedure. First, the quantity of enveloped α-amylase should have a total volume sufficient to allow the enveloped α-amylase to be spread throughout the dough mix. If the preparation of enveloped α-amylase is highly concentrated, additional oil may need to be added to the pre-mix before the enveloped α-amylase is added to the dough. Recipes and production processes may require specific modifications; however, good results generally can be achieved when 25% of the oil specified in a bread dough formula is held out of the dough and is used as a carrier for a concentrated enveloped α-amylase when added near the end of the mix cycle. In bread or other baked goods, recipes which have extremely low fat content (such as French-style breads), it has been found that an enveloped α-amylase mixture of approximately 1% of the dry flour weight is sufficient to admix the enveloped α-amylase properly with the dough, but the range of percentages that may work is extremely wide and depends on the formula, finished product, and production methodology requirements of the individual baker. Second, the enveloped α-amylase suspension should be added to the mix with enough time remaining in the mix cycle for complete mixture into the dough, but not so early that excessive mechanical action will strip the protective lipid coating from a large proportion of the enveloped α-amylase particles.

In another embodiment, bacterial α-amylase (BAA) is added to the lipid-coated particles comprising a B. licheniformis α-amylase variant. BAA reduces bread to a gummy mass due to its excessive thermostability and retained activity in the fully baked loaf of bread; however, when BAA is incorporated into the lipid-coated particles, substantial additional anti-staling protection is obtained, even at very low BAA dosage levels. For example, BAA dosages of 150 RAU (Reference Amylase Units) per 100 pounds of flour have been found to be effective. In one embodiment, between about 50 to 2000 RAU of BAA is added to the lipid-coated enzyme product. This low BAA dosage level, combined with the ability of the protective coating to keep enzyme in the fully-baked loaf from free contact with the starches (except when water vapor randomly releases the enzyme from its coating), helps to achieve very high levels of anti-staling activity without the negative side-effects of BAA.

It will be apparent to those skilled in the art that various modifications and variation can be made to the compositions and methods of using same without departing from the spirit or scope of the intended use. Thus, it is the modifications and variations provided they come within the scope of the appended claims and their equivalents.

### EXAMPLES

### Example 1

Modifying B. licheniformis α-amylase active site residues or charged residues on the enzyme surface to resemble corresponding residues in the high-performance Bacillus sp. no. 707 α-amylase will result in B. licheniformis α-amylase variants having a comparably higher performance. To this end, B. licheniformis α-amylase variants were designed by comparing the 3D structure of B. licheniformis α-amylase with the 3D structure of Bacillus sp. no. 707 α-amylase. Specifically, the BRAGI software was used to generate the alignment shown in FIG. 1. Modeling with BRAGI confirmed that the amino acid modifications would not alter significantly the conserved secondary and tertiary structural elements of the variant enzyme.

Model building was initiated using the 3D structure of a wild-type B. licheniformis α-amylase accessed from the RCSB Protein Data Bank as PDB ID No. 1BLI. This α-amylase has the amino acid sequence depicted in Example 1.1 below, where the sequence is altered to introduce the mutations M15T, W138Y, and M197T. In this manner, the starting B. licheniformis α-amylase has the same sequence as the α-amylase in PURASTAR® OxAm, or “Purastar α-amylase.” PURASTAR® OxAm is an oxidatively stable α-amylase for bleach-containing detergent formulations.

### Example 1.1

### Sequence of PURASTAR® OxAm α-amylase (SEQ ID NO 3)

ANLNGTLMQY FEWYTPNDGQ HWKRLQNDSA YLAEHGITAV
WIPPAYKGTS QADVGYGAYD
LYDLGEFHQK GTVRTKYGTK GELQSAIKSL HSRDINVYGD
VVINHKGGAD ATEDVTAVEV
DPADRNRVIS GEHLIKAYTH FHFPGRGSTY SDFKWHWYHF
DGTDWDESRK LNRIYKFQGK
AWDWEVSNEN GNYDYLTYAD IDYDHPDVAA EIKRWGTWYA
NELQLDGFRL DAVKHIKFSF
LRDWVNHVRE KTGKEMFTVA EYWQNDLGAL ENYLNKTNFN
HSVFDVPLHY QFHAASTQGG
GYDMRKLLNG TVVSKHPLKS VTFVDNHDTQ PGQSLESTVQ
TWFKPLAYAF ILTRESGYPQ
VFYGDMYGTK GDSQREIPAL KHKIEPILKA RKQYAYGAQH
DYFDHHDIVG WTREGDSSVA
NSGLAALITD GPGGAKRMYV GRQNAGETWH DITGNRSEPV
VINSEGWGEF HVNGGSVSIY
VQR

### Example 2

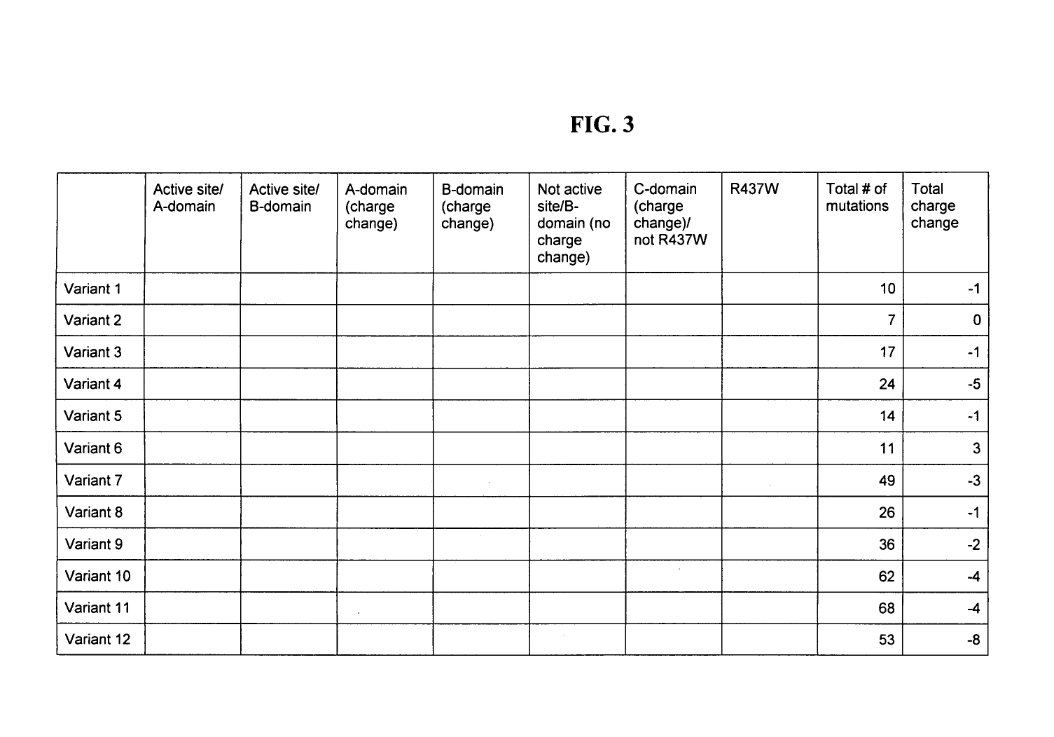

The PURASTAR® α-amylase was used as the basis for further modeling to design sequence modifications in specific domains, to active site residues, and/or to charged/uncharged residues on the enzyme surface. Twelve representative variant α-amylases designed using this approach are summarized in Table 1 below. In each case, the amino acid modification to the variant changes a residue to the corresponding residue on Bacillus sp. no. 707 α-amylase. The total number of amino acid changes and the effect of the changes on the overall charge of the enzyme are shown in the last row of the table. From the 3D structures of the α-amylases, it is known that all of the modifications that affect charge are to amino acids located on the surface of the enzyme. FIG. 3 provides a summary of the modifications, where groups of modifications are made to active site and/or charged residues in domains A, B, and/or C, as indicated.

TABLE 1

| | Active | | Charge | Variant | | | | | | | | | | | |
|---|---|---|---|---|---|---|---|---|---|---|---|---|---|---|---|
| Mutation | site? | Domain | Change | 1 | 2 | 3 | 4 | 5 | 6 | 7 | 8 | 9 | 10 | 11 | 12 |
| K23N | | A | −1 | | | | 1 | | | 1 | | | 1 | 1 | 1 |
| Q26R | | A | +1 | | | | 1 | | | 1 | | | 1 | 1 | 1 |
| A33K | | A | +1 | | | | 1 | | | 1 | | | 1 | 1 | 1 |
| T49A | Y | A | | 1 | | 1 | | | | | | 1 | 1 | 1 | |
| A52N | Y | A | | 1 | | 1 | | | | | | 1 | 1 | 1 | |
| H68N | | A | −1 | | | | 1 | | | 1 | | | 1 | 1 | 1 |
| E82Q | | A | +1 | | | | 1 | | | 1 | | | 1 | 1 | 1 |
| K88N | | A | −1 | | | | 1 | | | 1 | | | 1 | 1 | 1 |
| H91K | | A | | | | | 1 | | | 1 | | | 1 | 1 | 1 |
| R93N | | A | −1 | | | | 1 | | | 1 | | | 1 | 1 | 1 |
| D94G | | A | +1 | | | | 1 | | | 1 | | | 1 | 1 | 1 |
| D114L | | B | +1 | | | | | 1 | | 1 | 1 | 1 | 1 | 1 | 1 |
| T116R | | B | +1 | | | | | 1 | | 1 | 1 | 1 | 1 | 1 | 1 |
| D121N | | B | +1 | | | | | 1 | | 1 | 1 | 1 | 1 | 1 | 1 |
| A123N | | B | | | | | | | | | 1 | 1 | | 1 | |
| D124N | | B | +1 | | | | | 1 | | 1 | 1 | 1 | 1 | 1 | 1 |
| R127Q | | B | −1 | | | | | 1 | | 1 | 1 | 1 | 1 | 1 | 1 |
| V128E | | B | −1 | | | | | 1 | | 1 | 1 | 1 | 1 | 1 | 1 |
| I129V | | B | | | | | | | | | 1 | 1 | | 1 | |
| H133Y | | B | −1 | | | | | 1 | | 1 | 1 | 1 | 1 | 1 | 1 |
| L134T | | B | | | | | | | | | 1 | 1 | | 1 | |
| K136E | | B | −2 | | | | | 1 | | 1 | 1 | 1 | 1 | 1 | 1 |
| H140Y | | B | −1 | | | | | 1 | | 1 | 1 | 1 | 1 | 1 | 1 |
| H142D | | B | −2 | | | | | 1 | | 1 | 1 | 1 | 1 | 1 | 1 |
| S148N | | B | | | | | | | | | 1 | 1 | | 1 | |
| Y150H | | B | +1 | | | | | 1 | | 1 | 1 | 1 | 1 | 1 | 1 |
| D152N | | B | +1 | | | | | 1 | | 1 | 1 | 1 | 1 | 1 | 1 |
| H156R | | B | | | | | | 1 | | 1 | 1 | 1 | 1 | 1 | 1 |
| T163V | Y | B | | | 1 | 1 | | | | | 1 | 1 | 1 | 1 | 1 |
| E167Q | Y | B | +1 | | 1 | 1 | | | | | 1 | 1 | 1 | 1 | 1 |
| K170R | Y | B | | | 1 | 1 | | | | | 1 | 1 | 1 | 1 | 1 |
| 172+N | Y | B | | | 1 | 1 | | | | | 1 | 1 | 1 | 1 | |
| Q178R | | B | +1 | | | | | 1 | | 1 | 1 | 1 | 1 | 1 | 1 |
| A181G | | B | | | | | | | | | 1 | 1 | | 1 | |
| S187D | Y | B | −1 | | 1 | 1 | | | | | 1 | 1 | 1 | 1 | 1 |
| N188T | Y | B | | | 1 | 1 | | | | | 1 | 1 | 1 | 1 | 1 |
| N190F | Y | B | | | 1 | 1 | | | | | 1 | 1 | 1 | 1 | 1 |
| K213R | | A | | | | | 1 | | | 1 | | | 1 | 1 | 1 |
| R214N | | A | −1 | | | | 1 | | | 1 | | | 1 | 1 | 1 |
| E222T | | A | +1 | | | | 1 | | | 1 | | | 1 | 1 | 1 |
| F238Y | Y | A | | 1 | | 1 | | | | | | 1 | | | 1 |
| E250S | | A | +1 | | | | 1 | | | 1 | | | 1 | 1 | 1 |
| K251A | | A | −1 | | | | 1 | | | 1 | | | 1 | 1 | 1 |
| E255N | | A | +1 | | | | 1 | | | 1 | | | 1 | 1 | 1 |
| Y262F | Y | A | | 1 | | 1 | | | | | | 1 | 1 | 1 | 1 |
| Q264K | Y | A | +1 | 1 | | 1 | | | | | | 1 | 1 | 1 | 1 |
| H293Y | Y | A | −1 | 1 | | 1 | | | | | | 1 | 1 | 1 | 1 |
| T297K | Y | A | +1 | 1 | | 1 | | | | | | 1 | 1 | 1 | 1 |
| R305Q | | A | −1 | | | | 1 | | | 1 | | | 1 | 1 | 1 |
| K306N | | A | −1 | | | | 1 | | | 1 | | | 1 | 1 | 1 |
| K319H | | A | | | | | 1 | | | 1 | | | 1 | 1 | 1 |
| G332E | Y | A | −1 | 1 | | 1 | | | | | | 1 | 1 | 1 | 1 |
| Q333E | Y | A | −1 | 1 | | 1 | | | | | | 1 | 1 | 1 | 1 |
| S334A | Y | A | | 1 | | 1 | | | | | | 1 | 1 | 1 | 1 |
| Q340E | | A | −1 | | | | 1 | | | 1 | | | 1 | 1 | 1 |
| T341E | | A | −1 | | | | 1 | | | 1 | | | 1 | 1 | 1 |
| IPTHGV-_{—} | | A | | | | | 1 | | | 1 | | | 1 | 1 | 1 |
| K389E | | A | −2 | | | | 1 | | | 1 | | | 1 | 1 | 1 |
| K392Q | | A | −1 | | | | 1 | | | 1 | | | 1 | 1 | 1 |
| Q393K | | A | +1 | | | | 1 | | | 1 | | | 1 | 1 | 1 |
| A398R | | C | +1 | | | | | | 1 | 1 | | | | | |
| H400N | | C | −1 | | | | | | 1 | 1 | | | | | |
| D416N | | C | +1 | | | | | | 1 | 1 | | | 1 | 1 | |
| V419H | | C | +1 | | | | | | 1 | 1 | | | 1 | 1 | |
| R437W | | C | −1 | | | | | | 1 | 1 | | | 1 | 1 | 1 |
| N444K | | C | +1 | | | | | | 1 | 1 | | | 1 | 1 | |
| E447Q | | C | +1 | | | | | | 1 | 1 | | | 1 | 1 | |
| H450S | | C | −1 | | | | | | 1 | 1 | | | 1 | 1 | |
| E458G | | C | +1 | | | | | | 1 | 1 | | | 1 | 1 | |
| E469N | | C | +1 | | | | | | 1 | 1 | | | 1 | 1 | |
| H471S | | C | −1 | | | | | | 1 | 1 | | | 1 | 1 | |

| | | | | | | | | | | | | |
|---|---|---|---|---|---|---|---|---|---|---|---|---|
| Total modifications | 10 | 7 | 17 | 24 | 14 | 11 | 49 | 26 | 36 | 62 | 68 | 53 |
| (charge change): | (−1) | (0) | (−1) | (−5) | (−1) | (3) | (−3) | (−1) | (−2) | (−4) | (−4) | (−8) |

### Example 3

Three active site variants were modeled, each with various changes to active site residues. Active Site Variant 1 contains 10 active site residue modifications (see Table 1), all of which are in domain A. Active Site Variant 2 contains 7 active site residue modifications, all of which are in domain B (see Table 1). Active Site Variant 3 combines the amino acid modifications from Variants 1 and 2. The complete sequences of Variants 1, 2 and 3 are shown below.

### Example 3.1

### Active Site Variant 1 (Domain A) (SEQ ID NO: 4)

ANLNGTLMQY FEWYTPNDGQ HWKRLQNDSA YLAEHGITAV
WIPPAYKGAS QNDVGYGAYD LYDLGEFHQK GTVRTKYGTK
GELQSAIKSL HSRDINVYGD VVINHKGGAD ATEDVTAVEV
DPADRNRVIS GEHLIKAYTH FHFPGRGSTY SDFKWHWYHF
DGTDWDESRK LNRIYKFQGK AWDWEVSNEN GNYDYLTYAD
IDYDHPDVAA EIKRWGTWYA NELQLDGFRL DAVKHIKYSF
LRDWVNHVRE KTGKEMFTVA EFWKNDLGAL ENYLNKTNFN
HSVFDVPLHY QFYAASKQGG GYDMRKLLNG TVVSKHPLKS
VTFVDNHDTQ PEEALESTVQ TWFKPLAYAF ILTRESGYPQ
VFYGDMYGTK GDSQREIPAL KHKIEPILKA RKQYAYGAQH
DYFDHHDIVG WTREGDSSVA NSGLAALITD GPGGAKRMYV
GRQNAGETWH DITGNRSEPV VINSEGWGEF HVNGGSVSIY
VQR

### Example 3.2

### Active Site Variant 2 (Domain B) (SEQ ID NO:5)

ANLNGTLMQY FEWYTPNDGQ HWKRLQNDSA YLAEHGITAV
WIPPAYKGTS QADVGYGAYD LYDLGEFHQK GTVRTKYGTK
GELQSAIKSL HSRDINVYGD VVINHKGGAD ATEDVTAVEV
DPADRNRVIS GEHLIKAYTH FHFPGRGSTY SDFKWHWYHF
DGVDWDQSRR LNRIYKFQGK AWDWEVDTEF GNYDYLTYAD
IDYDHPDVAA EIKRWGTWYA NELQLDGFRL DAVKHIKFSF
LRDWVNHVRE KTGKEMFTVA EYWQNDLGAL ENYLNKTNFN
HSVFDVPLHY QFHAASTQGG GYDMRKLLNG TVVSKHPLKS
VTFVDNHDTQ PGQSLESTVQ TWFKPLAYAF ILTRESGYPQ
VFYGDMYGTK GDSQREIPAL KHKIEPILKA RKQYAYGAQH
DYFDHHDIVG WTREGDSSVA NSGLAALITD GPGGAKRMYV
GRQNAGETWH DITGNRSEPV VINSEGWGEF HVNGGSVSIY
VQR

### Example 3.3

### Active Site Variant 3 (Domains A and B) (SEQ ID NO: 6)

ANLNGTLMQY FEWYTPNDGQ HWKRLQNDSA YLAEHGITAV
WIPPAYKGAS QNDVGYGAYD LYDLGEFHQK GTVRTKYGTK
GELQSAIKSL HSRDINVYGD VVINHKGGAD ATEDVTAVEV
DPADRNRVIS GEHLIKAYTH FHFPGRGSTY SDFKWHWYHF
DGVDWDQSRR LNNRIYKFQG KAWDWEVDTEF GNYDYLTYAD
IDYDHPDVAA EIKRWGTWYA NELQLDGFRL DAVKHIKYSF
LRDWVNHVRE KTGKEMFTVA EFWKNDLGAL ENYLNKTNFN
HSVFDVPLHY QFYAASKQGG GYDMRKLLNG TVVSKHPLKS
VTFVDNHDTQ PEEALESTVQ TWFKPLAYAF ILTRESGYPQ
VFYGDMYGTK GDSQREIPAL KHKIEPILKA RKQYAYGAQH
DYFDHHDIVG WTREGDSSVA NSGLAALITD GPGGAKRMYV
GRQNAGETWH DITGNRSEPV VINSEGWGEF HVNGGSVSIY
VQR

### Example 4

Variants also were modeled that contained modifications affecting the charge distribution on the enzyme surface but not the composition of active site residues. Charge Variant 4 contains modifications to charged residues in domain A, Charge Variant 5 contains modifications to charged residues in domain B, and Charge Variant 6 contains modifications to charged residues in domain C. Charge Variant 7 contains all the changes made in Variants 4, 5, and 6. The complete sequences of Variants 4-7 are shown below.

### Example 4.1

### Charge Variant 4 (Domain A) (SEQ ID NO:7)

ANLNGTLMQY FEWYTPNDGQ HWNRLRNDSA YLKEHGITAV
WIPPAYKGTS QADVGYGAYD LYDLGEFNQK GTVRTKYGTK
GQLQSAINSL KSNGINVYGD VVINHKGGAD ATEDVTAVEV
DPADRNRVIS GEHLIKAYTH FHFPGRGSTY SDFKWHWYHF
DGTDWDESRK LNRIYKFQGK AWDWEVSNEN GNYDYLTYAD
IDYDHPDVAA EIRNWGTWYA NTLQLDGFRL DAVKHIKFSF
LRDWVNHVRS ATGKNMFTVA EYWQNDLGAL ENYLNKTNFN
HSVFDVPLHY QFHAASTQGG GYDMQNLLNG TVVSKHPLHS
VTFVDNHDTQ PGQSLESTVE EWFKPLAYAF ILTRESGYPQ
VFYGDMYGIP THGV---PAL KHKIEPILEA RQKYAYGAQH
DYFDHHDIVG WTREGDSSVA NSGLAALITD GPGGAKRMYV
GRQNAGETWH DITGNRSEPV VINSEGWGEF HVNGGSVSIY
VQR

### Example 4.2

### Charge Variant 5 (Domain B) (SEQ ID NO:8)

ANLNGTLMQY FEWYTPNDGQ HWKRLQNDSA YLAEHGITAV
WIPPAYKGTS QADVGYGAYD LYDLGEFHQK GTVRTKYGTK
GELQSAIKSL HSRDINVYGD VVINHKGGAD ATELVRAVEV
NPANRNQEIS GEYLIEAYTY FDFPGRGSTH SNFKWRWYHF
DGTDWDESRK LNRIYKFRGK AWDWEVSNEN GNYDYLTYAD
IDYDHPDVAA EIKRWGTWYA NELQLDGFRL DAVKHIKFSF
LRDWVNHVRE KTGKEMFTVA EYWQNDLGAL ENYLNKTNFN
HSVFDVPLHY QFHAASTQGG GYDMRKLLNG TVVSKHPLKS
VTFVDNHDTQ PGQSLESTVQ TWFKPLAYAF ILTRESGYPQ
VFYGDMYGTK GDSQREIPAL KHKIEPILKA RKQYAYGAQH
DYFDHHDIVG WTREGDSSVA NSGLAALITD GPGGAKRMYV
GRQNAGETWH DITGNRSEPV VINSEGWGEF HVNGGSVSIY
VQR

### Example 4.3

### Charge Variant 6 (Domain C) (SEQ ID NO:9)

ANLNGTLMQY FEWYTPNDGQ HWKRLQNDSA YLAEHGITAV
WIPPAYKGTS QADVGYGAYD LYDLGEFHQK GTVRTKYGTK
GELQSAIKSL HSRDINVYGD VVINHKGGAD ATEDVTAVEV
DPADRNRVIS GEHLIKAYTH FHFPGRGSTY SDFKWHWYHF
DGTDWDESRK LNRIYKFQGK AWDWEVSNEN GNYDYLTYAD
IDYDHPDVAA EIKRWGTWYA NELQLDGFRL DAVKHIKFSF
LRDWVNHVRE KTGKEMFTVA EYWQNDLGAL ENYLNKTNFN
HSVFDVPLHY QFHAASTQGG GYDMRKLLNG TVVSKHPLKS
VTFVDNHDTQ PGQSLESTVQ TWFKPLAYAF ILTRESGYPQ
VFYGDMYGTK GDSQREIPAL KHKIEPILKA RKQYAYGRQN
DYFDHHDIVG WTREGNSSHA NSGLAALITD GPGGAKWMYV
GRQKAGQTWS DITGNRSGPV VINSEGWGNF SVNGGSVSIY
VQR

### Example 4.4

### Charge Variant 7 (All Domains) (SEQ ID NO:10)

ANLNGTLMQY FEWYTPNDGQ HWNRLRNDSA YLKEHGITAV
WIPPAYKGTS QADVGYGAYD LYDLGEFNQK GTVRTKYGTK
GQLQSAINSL KSNGINVYGD VVINHKGGAD ATELVRAVEV
NPANRNQEIS GEYLIEAYTY FDFPGRGSTH SNFKWRWYHF
DGTDWDESRK LNRIYKFRGK AWDWEVSNEN GNYDYLTYAD
IDYDHPDVAA EIRNWGTWYA NTLQLDGFRL DAVKHIKFSF
LRDWVNHVRS ATGKNMFTVA EYWQNDLGAL ENYLNKTNFN
HSVFDVPLHY QFHAASTQGG GYDMQNLLNG TVVSKHPLHS
VTFVDNHDTQ PGQSLESTVE EWFKPLAYAF ILTRESGYPQ
VFYGDMYGIP THGV---PAL KHKIEPILEA RQKYAYGRQN
DYFDHHDIVG WTREGNSSHA NSGLAALITD GPGGAKWMYV
GRQKAGQTWS DITGNRSGPV VINSEGWGNF SVNGGSVSIY
VQR

### Example 5

Variants also were modeled that contained various combinations of modifications to surface charges and active site residues in various domains. Variant 8 contains all the modifications to domain B; Variant 9 contains all the modifications to domain B and the active site changes in domain A; Variant 10 contains the modifications to charged residues in all the domains and the active site changes in domains A and B. Variant 11 contains all the modifications to the surface charge of the enzyme, to the active site, and to domain B. Variant 12 contains all the charge changes to domains A and B, all the active site changes to domains A and B, and the R437W substitution. The complete sequence of Variants 8-12 are shown below.

### Example 5.1

### Variant 8 (All Domain B Modifications) (SEQ ID NO:11)

ANLNGTLMQY FEWYTPNDGQ HWKRLQNDSA YLAEXGITAV
WIPPAYKGTS QADVGYGAYD LYDLGEFHQK GTVRTKYGTK
GELQSAIKSL HSRDINVYGD VVINHKGGAD ATELVRAVEV
NPNNRNQEVS GEYTIEAYTY FDFPGRGNTH SNFKWRWYHF
DGVDWDQSRR LNNRIYKFRGK AWDWEVDTEF GNYDYLTYAD
IDYDHPDVAA EIKRWGTWYA NELQLDGFRL DAVKHIKFSF
LRDWVNHVRE KTGKEMFTVA EYWQNDLGAL ENYLNKTNFN
HSVFDVPLHY QFHAASTQGG GYDMRKLLNG TVVSKHPLKS
VTFVDNHDTQ PGQSLESTVQ TWFKPLAYAF ILTRESGYPQ
VFYGDMYGTK GDSQREIPAL KHKIEPILKA RKQYAYGAQH
DYFDHHDIVG WTREGDSSVA NSGLFALITD GPGGAKRMYV
GRQNAGETWH DITGNRSEPV VINSEGWGEF HVNGGSVSIY
VQR

### Example 5.2

### Variant 9 (All Domain B Modifications and Domain A Active Site Modifications) (SEQ ID NO:12)

ANLNGTLMQY FEWYTPNDGQ HWKRLQNDSA YLAEHGITAV
WIPPAYKGAS QNDVGYGAYD LYDLGEFHQK GTVRTKYGTK
GELQSAIKSL HSRDINVYGD VVINHKGGAD ATELVRAVEV
NPNNRNQEVS GEYTIEAYTY FDFPGRGNTH SNFKWRWYHF
DGVDWDQSRR LNNRIYKFRGK GWDWEVDTEF GNYDYLTYAD
IDYDHPDVAA EIKRWGTWYA NELQLDGFRL DAVKHIKYSF
LRDWVNHVRE KTGKEMFTVA EFWKNDLGAL ENYLNKTNFN
HSVFDVPLHY QFYAASKQGG GYDMRKLLNG TVVSKHPLKS
VTFVDNHDTQ PEEALESTVQ TWFKPLAYAF ILTRESGYPQ
VFYGDMYGTK GDSQREIPAL KHKIEPILKA RKQYAYGAQH
DYFDHHDIVG WTREGDSSVA NSGLAALITD GPGGAKRMYV
GRQNAGETWH DITGNRSEPV VINSEGWGEF HVNGGSVSIY
VQR

### Example 5.3

### Variant 10 (All Charge Modifications and Active Site Modifications in Domain A and B) (SEQ ID NO:13)

ANLNGTLMQY FEWYTPNDGQ HWNRLRNDSA YLKEHGITAV
WIPPAYKGAS QNDVGYGAYD LYDLGEFNQK GTVRTKYGTK
GQLQSAINSL KSNGINVYGD VVINHKGGAD ATELVRAVEV
NPANRNQEIS GEYLIEAYTY FDFPGRGSTH SNFKWRWYHF
DGVDWDQSRR LNNRIYKFRGK AWDWEVDTEF GNYDYLTYAD
IDYDHPDVAA EIRNWGTWYA NTLQLDGFRL DAVKHIKYSF
LRDWVNHVRS ATGKNMFTVA EFWKNDLGAL ENYLNKTNFN
HSVFDVPLHY QFYAASKQGG GYDMQNLLNG TVVSKHPLHS
VTFVDNHDTQ PEEALESTVE EWFKPLAYAF ILTRESGYPQ
VFYGDMYGIP THGV---PAL KHKIEPILEA RQKYAYGRQN
DYFDHHDIVG WTREGNSSHA NSGLAALITD GPGGAKWMYV
GRQKAGQTWS DITGNRSGPV VINSEGWGNF SVNGGSVSIY
VQR

### Example 5.4

### Variant 11 (All Charge Changes, Active Site Changes, and Domain B Changes) (SEQ ID NO:14)

ANLNGTLMQY FEWYTPNDGQ HWNRLRNDSA YLKEHGITAV
WIPPAYKGAS QNDVGYGAYD LYDLGEFNQK GTVRTKYGTK
GQLQSAINSL KSNGINVYGD VVINHKGGAD ATELVRAVEV
NPNNRNQEVS GEYTIEAYTY FDFPGRGNTH SNFKWRWYHF
DGVDWDQSRR LNNRIYKFRGK GWDWEVDTE FGNYDYLTYAD
IDYDHPDVAA EIRNWGTWYA NTLQLDGFRL DAVKHIKYSF
LRDWVNHVRS ATGKNMFTVA EFWKNDLGAL ENYLNKTNFN
HSVFDVPLHY QFYAASKQGG GYDMQNLLNG TVVSKHPLHS
VTFVDNHDTQ PEEALESTVE EWFKPLAYAF ILTRESGYPQ
VFYGDMYGIP THGV---PAL KHKIEPILEA RQKYAYGRQN
DYFDHHDIVG WTREGNSSHA NSGLAALITD GPGGAKWMYV
GRQKAGQTWS DITGNRSGPV VINSEGWGNF SVNGGSVSIY
VQR

### Example 5.5

### Variant 12 (All Domain A and B Charge Changes, Domain A and B Active Site Changes, and R437W) (SEQ ID NO:15)

ANLNGTLMQY FEWYTPNDGQ HWNRLRNDSA YLKEHGITAV
WIPPAYKGAS QNDVGYGAYD LYDLGEFNQK GTVRTKYGTK
GQLQSAINSL KSNGINVYGD VVINHKGGAD ATELVRAVEV
NPANRNQEIS GEYLIEAYTY FDFPGRGSTH SNFKWRWYHF
DGVDWDQSRR LNRIYKFRGK AWDWEVDTEF GNYDYLTYAD
IDYDHPDVAA EIRNWGTWYA NTLQLDGFRL DAVKHIKYSF
LRDWVNHVRS ATGKNMFTVA EFWKNDLGAL ENYLNKTNFN
HSVFDVPLHY QFYAASKQGG GYDMQNLLNG TVVSKHPLHS
VTFVDNHDTQ PEEALESTVE EWFKPLAYAF ILTRESGYPQ
VFYGDMYGIP THGV---PAL KHKIEPILEA RQKYAYGAQH
DYFDHHDIVG WTREGDSSVA NSGLAALITD GPGGAKWMYV
GRQNAGQTWH DITGNRSEPV VINSEGWGEF HVNGGSVSIY
VQR

### Example 6

### Construction of Expression Vector Containing OxAm Variants

This example describes how the expression vector was constructed.

Synthetic genes for 12 OxAm variants were synthesized by GeneArt, Inc. (Toronto, Canada) and cloned into PCR-Script plasmid. The gene constructs were obtained as 0.2 μg/μL of plasmid DNA. Genes for variants 1, 5, 6, 7, 8, 10, 11, and 12 were amplified from GeneArt PCR-Script plasmid using PureTaq beads from Amersham using the following primers:

| | | |
|---|---|---|
| pGeneart-F1: | CTCTTCGCTATTACGCCAGCTG | (SEQ ID NO: 31) |
| pGeneart-R1 | GCTATGACCATGATTACGCCAAG | (SEQ ID NO: 32) |

Genes for variants 2, 3, and 4 were amplied using the following primers:

| | |
|---|---|
| pGeneart-F2 GCCATTCAGGCTGCGCAACTGT | (SEQ ID NO: 33) |
| pGeneart-R2 TGCTTCCGGCTCGTATGTTGTG | (SEQ ID NO: 34) |

PCR conditions were as follows: 95° C. for 2 min, 1×, followed by 30 cycles of 95° C. 1 min, 52° C., 1 min, 72° C. for 1 min 30 sec, followed by 7 min at 72° C. All PCR products were purified using Qiagen Qiaquick columns and resuspended in 504 of milliQ water. 50 μL of eluted DNA was cut with HpaI (Roche), purified, and resuspended in 904 of milliQ water. The resuspended DNA was subsequently cut with PstI (Roche) in a 1004 final volume reaction, purified and resuspended in 30 μL of milliQ water. 2 μL of eluted DNA was ligated with 1 μL of B. subtilis vector pHPLT (10-20 ng/μL). The vector pHPLT is described in U.S. Pat. No. 6,566,112.

The ligation mixtures were transformed into Bacillus subtilis strain (genotype: ΔaprE, ΔnprE, Δepr, ΔispA, Δbpr, degU^{Hy}32, oppA, ΔspoIIE3501, amyE::xylRPxylAcomK-ermC) competent cells. WW120 cells have a competency gene (comK), which is placed under a xylose inducible promoter, thus xylose can be used to induce competency for DNA binding and uptake. Colony PCR using PureTaq beads from Amersham was performed on the transformants by resuspending the colonies in 20 μL of water and using 2 μL of cells with 0.5 μL of pHPLT-F1 and R1 primers in a 25 μL reaction:

| | |
|---|---|
| PHPLT-F1 TACATATGAGTTATGCAGTTTG | (SEQ ID NO: 35) |
| PHPLT-R1 GTTATGAGTTAGTTCAAATTCG | (SEQ ID NO: 36) |

Each construct was sequenced using pHPLT-seqF1 and seqR1 primers at Sequetech

| | |
|---|---|
| pHPLT-SEQF1 GGAGGAGAATCATGAAAC | (SEQ ID NO: 37) |
| pHPLT-SEQR1 TTATCCTTTACCTTGTCTC | (SEQ ID NO: 38) |

Single colonies for each variant, were grown in 4 mL of Luria Broth (LB)+10 ppm neomycin and stored as glycerol stocks.

For protein expression, Bacillus subtilis strain WW120 cells containing genes for OxAm variants were inoculated into 25 mL of cultivation medium 1 (M1) and 2 (M2) (described below) containing 10 μg/mL neomycin and cultured for about 64 hours at 37° C. @ 250 rpm. Two mL of culture was centrifuged at 25,000×g and supernatant was harvested, filtered and stored at 0° C. to 4° C. M1 was an enriched semi-defined media based on MOPs buffer, with urea as the major nitrogen source, glucose as the main carbon source, and supplemented with 1% soytone for robust cell growth. M2 was similar to cultivation medium 1 except it lacks soytone, contains less glucose, and is supplemented with 3.5% Maltrin-150 (Grain Processing Corp., Iowa).

### Example 7

### Variant Wash Performance

This example examines the performance characteristics of the variants described herein. For quantitative protein determination, 30 μL of OxAm wild type and variant cultures were analyzed by running on 10% acrylamide gel (MES buffer) and staining with Coomassie Blue R250 dye, followed by quantitation using Scion Image software (Scion Corp., Frederick, Md., version Beta 4.03), including a sample of purified OxAm protein as standard.

To determine the stain removal performance of OxAm and OxAm variants, CS-26 corn-starch colored swatches (TestFabrics Inc., West Pittiston, Pa.) were cut to 0.25 inches and added to 96-well plates. IEC“A” detergent (a standard non-phosphate detergent) was prepared fresh at a concentration of 8 g/L and filtered. 150 ppm of water hardness was added to the detergent. 200 μL aliquots of this detergent mixture were added to the swatches followed by addition of OxAm parent or variant protein samples to yield 0.5 and 2 ppm concentrations. The plates were shaken and incubated at 20° C. for 60 minutes at 750 rpm on an Eppendorf Thermomixer set. Aliquots of 150 μl. were transferred to a fresh plate and the optical density was determined at 488 nm using a microplate reader.

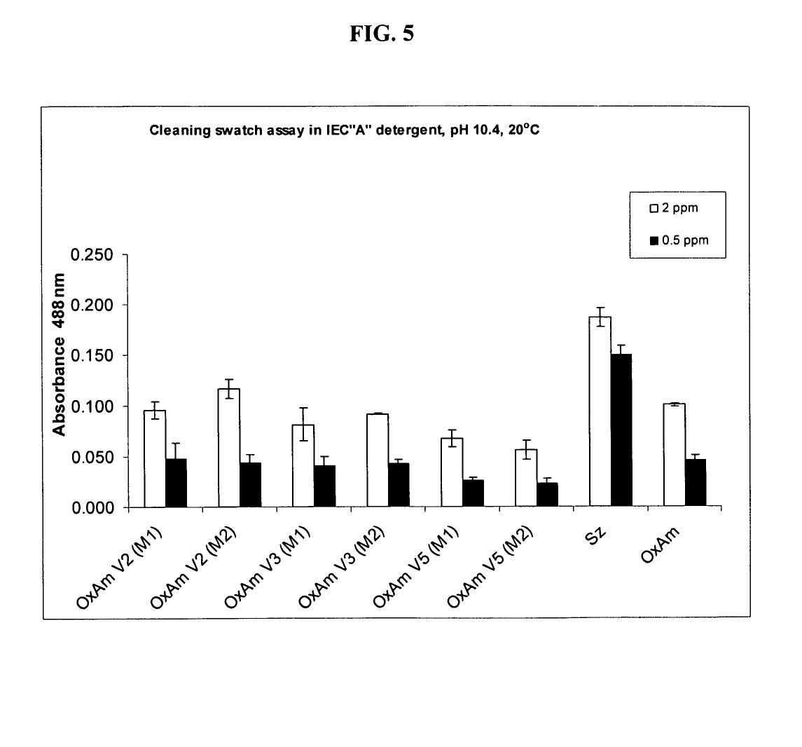

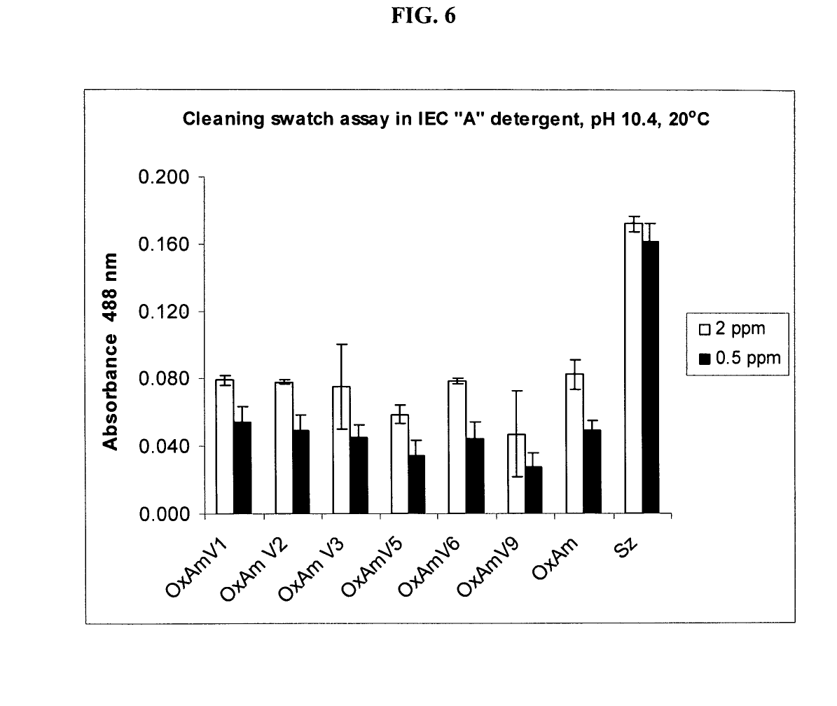

Experiments were conducted to determine stain removal performance of OxAm parent and OxAm variant proteins at pH 10.4 using the Cleaning Swatch Assay for Stain Removal Performance as described above. FIG. 5 shows the performance of OxAm variants V2, V3, and V5 grown in either cultivation medium 1 (M1) or cultivation medium 2 (M2), and compared to OxAm parent and Stainzyme (Sz). FIG. 6 shows the performance of OxAm variants V1, V2, V3, V5, V6, and V9 grown in cultivation medium 1 (M1), and compared to OxAm parent and Stainzyme (Sz) grown in the same conditions. Cleaning activity was measured by color release from CS-26 swatches following 60 min incubation at pH 10.4 and 20° C.

All references cited above are herein incorporated by reference in their entirety for all purposes.

1. An isolated nucleic acid encoding a variant of SEQ ID NO:1, wherein the variant comprises at least one amino acid substitution, insertion, or deletion compared to SEQ ID NO:1, and wherein the encoded variant exhibits α-amylase activity.

2. The nucleic acid of claim 1, wherein the at least one amino acid substitution, insertion, or deletion results in the encoded variant comprising an amino acid residue that corresponds to an amino acid residue of the Bacillus sp. no. 707 α-amylase set forth in SEQ ID NO:2.

3. The nucleic acid of claim 1, wherein the at least one amino acid substitution, insertion, or deletion is made to a charged residue located on the surface of the encoded variant.

4. The nucleic acid of claim 1, wherein the at least one amino acid substitution, insertion, or deletion is made to an active site amino acid residue.

5. The nucleic acid of claim 1, wherein the at least one amino acid substitution, insertion, or deletion is made to an amino acid residue other than the residue at position 1.

6. The nucleic acid of claim 1, wherein the variant comprises a domain A extending from residues 2-105 and residues 208-396, domain B extending from residues 106-207; and domain C extending from residue 397 to the C terminus of said encoded variant.

7. The nucleic acid of claim 6, wherein the encoded variant has at least one amino acid substitution, insertion, or deletion in domain A.

8. The nucleic acid of claim 6, wherein the encoded variant has at least one amino acid substitution, insertion, or deletion in domain B.

9. The nucleic acid of claim 6, wherein the encoded variant has at least one amino acid substitution, insertion, or deletion in domain C.

10. The nucleic acid of claim 1, wherein the encoded variant comprises at least two amino acids that are substituted, inserted, or deleted.

11. The nucleic acid of claim 10, wherein the encoded variant comprises at least five amino acids that are substituted, inserted, or deleted.

12. The nucleic acid of claim 11, wherein the encoded variant comprises at least ten amino acids that are substituted, inserted, or deleted.

13. The nucleic acid of claim 12, wherein the encoded variant comprises between 11 and 30 amino acid substitutions, insertions, or deletions.

14. The nucleic acid of claim 12, wherein the encoded variant comprises between 11 and 70 amino acid substitutions, insertions, or deletions.

15. The nucleic acid of claim 1, where the encoded variant has the amino acid sequence shown in any one of the polypeptides of SEQ ID NOS:4 to 15.

16. The nucleic acid of claim 1, where the encoded variant comprises one or more of the following amino acid substitutions, insertions, or deletions: K23N; Q26R; A33K; T49A; A52N; H68N; E82Q; K88N; H91K; R93N; D94G; D114L; T116R; D121N; A123N; D124N; R127Q; V128E; 1129V; H133Y; L134T; K136E; H140Y; H142D; S148N; Y150H; D152N; H156R; T163V; E167Q; K170R; insertion of N at position 172; Q178R; A181G; S187D; N188T; N190F; K213R; R214N; E222T; F238Y; E250S; K251A; E255N; Y262F; Q264K; H293Y; T297K; R305Q; K306N; K319H; G332E; Q333E; S334A; Q340E; T341E; substitution or deletion of residues 369-377 from TKGDSQREI to IPTHGV---, where the hyphens represent deletions; K389E; K392Q; Q393K; A398R; H400N; D416N; V419H; R437W; N444K; E447Q; H450S; E458G; E469N; or H471S.

17. An isolated host cell comprising the nucleic acid of claim 1.

18. A vector comprising the nucleic acid of claim 1.

19. A host cell comprising the vector of claim 18.

20. The host cell of any of claim 17, wherein the cell is a microorganism.

21. The host cell of claim 20, wherein the microorganism is a bacterium or a fungus.

22. The isolated host cell of claim 21, wherein the bacterium is a Gram positive bacterium selected from the group consisting of Bacillus subtilis, B. licheniformis, B. lentus, B. brevis, B. stearothermophilus, B. alkalophilus, B. amyloliquefaciens, B. coagulans, B. circulans, B. lautus, B. thuringiensis, Streptomyces lividans, or S. murinus; or a Gram negative bacterium, wherein said Gram negative bacterium is Escherichia coli or a Pseudomonas sp.

23. A variant encoded by the nucleic acid of claim 1.

24. A manual or automatic dishwashing composition comprising the variant of claim 23.

25. The manual or automatic dishwashing composition of claim 24, further comprising one or more of a surfactant, detergent builder, a complexing agent, a polymer, a bleaching system, a stabilizer, a foam booster, a suds suppressor, an anti-corrosion agent, a soil-suspending agent, an anti-soil redeposition agent, a dye, a bactericide, a hydrotope, a tarnish inhibitor, and a perfume.

26. A method of cleaning dishes comprising administering the manual or automatic dishwashing composition of claim 24 for a time sufficient to clean said dishes.

27. A detergent additive comprising the variant of claim 23.

28. A laundry detergent comprising the detergent additive of claim 27, and further comprising one or more of a surfactant, detergent builder, a complexing agent, a polymer, a bleaching system, a stabilizer, a foam booster, a suds suppressor, an anti-corrosion agent, a soil-suspending agent, an anti-soil redeposition agent, a dye, a bactericide, a hydrotope, an optical brightener, a fabric conditioner, and a perfume.

29. Use of the detergent additive of claim 27 for laundry washing or dishwashing.

30. Use of the variant of claim 23 for laundry washing or dishwashing.

31. A detergent additive comprising the variant of claim 23, optionally in the form of a non-dusting granulate, microgranulate, stabilized liquid, or protected enzyme.

32. The detergent additive of claim 31, wherein the detergent additive further comprises an enzyme selected from the group consisting of: a cellulase, a protease, an aminopeptidase, an amylase, a carbohydrase, a carboxypeptidase, a catalase, a chitinase, a cutinase, a cyclodextrin glucanotransferase, a deoxyribonuclease, an esterase, an α-galactosidase, a β-galactosidase, a glucoamylase, α-glucosidase, a β-glucosidase, a haloperoxidase, an invertase, a laccase, a lipase, a mannosidase, an oxidase, a pectinolytic enzyme, a peptidoglutaminase, a peroxidase, a phytase, a polyphenoloxidase, a proteolytic enzyme, a ribonuclease, a transglutaminase, a xylanase, a pullulanase, an isoamylase, a carrageenase, or any combination thereof.

33. The detergent additive of claim 32, wherein the amylase is another α-amylase, a β-amylase, an isoamylase, or a glucoamylase.

34. A detergent composition comprising the detergent additive of claim 31.

35. A detergent composition comprising the variant of claim 23.

36. The detergent composition of claim 35, further comprising an enzyme from the group consisting of: a cellulase, a protease, an aminopeptidase, an amylase, a carbohydrase, a carboxypeptidase, a catalase, a chitinase, a cutinase, a cyclodextrin glucanotransferase, a deoxyribonuclease, an esterase, an α-galactosidase, a β-galactosidase, a glucoamylase, an α-glucosidase, a β-glucosidase, a haloperoxidase, an invertase, a laccase, a lipase, a mannosidase, an oxidase, a pectinolytic enzyme, a peptidoglutaminase, a peroxidase, a phytase, a polyphenoloxidase, a proteolytic enzyme, a ribonuclease, a transglutaminase, a xylanase, a pullulanase, an isoamylase, a carrageenase, or any combination thereof.

37. A textile desizing composition comprising the variant of claim 23 in an aqueous solution, and optionally comprising another enzyme.

38. A method of desizing a textile comprising administering the desizing composition of claim 37 for a time sufficient to desize said textile.

39. Use of the variant of claim 23 for textile desizing.

40. A starch processing composition comprising the variant of claim 23 in an aqueous solution.

41. The starch processing composition of claim 40, further comprising a glucoamylase, an isoamylase, a pullulanase, phytase or a combination thereof.

42. A method of processing a starch comprising administering the composition of claim 40 for a time sufficient to process said starch.

43. A biofilm hydrolyzing composition comprising the variant of claim 23 in a solution or gel, and optionally further comprising a cellulase, a hemicellulase, a xylanase, a lipase, a protease, a pectinase, an antimicrobial agent, or any combination thereof.

44. A method of hydrolyzing a biofilm comprising administering the composition of claim 43 for a period sufficient to process said biofilm.

45. A composition for saccharifying starch comprising the variant of claim 23 in a solution.

46. A method of saccharifying starch comprising administering the composition of claim 45 for a period sufficient to saccharify said starch.

47. A composition for liquefying starch comprising the variant of claim 23 in a solution.

48. A method of liquefying a starch comprising administering the composition of claim 47 for a period sufficient to liquefy said starch.

49. A baking composition comprising the variant of claim 23 in a solution or in a gel.

50. A method of baking comprising administering the baking composition of claim 49.

51. A method of making a B. licheniformis α-amylase variant, comprising
(1) comparing the structure of a wild-type B. licheniformis α-amylase to a model α-amylase that possesses at least one preferred property relative to said wild-type B. licheniformis α-amylase;
(2) identifying at least one amino acid or structural region of the wild-type B. licheniformis α-amylase that is structurally conserved with the model α-amylase;
(3) constructing a variant of the wild-type B. licheniformis α-amylase, which is modified in the amino acid residue or structural region identified in step (2) above; and
(4) testing the variant to determine whether the at least one preferred property is conferred upon the variant,
wherein the variant has at least one altered property compared to the wild-type B. licheniformis α-amylase.

52. The method of claim 51, where the model α-amylase is a Bacillus sp. no. 707 α-amylase.
